(19) United States
(12) Patent Application Publication
(10) Pub. No.: US 2022/0241222 A1
(43) Pub. Date: Aug. 4, 2022

(54) ATOMOXETINE HYDROCHLORIDE EXTENDED RELEASE COMPOSITIONS AND METHODS OF USE

(71) Applicant: CELISTA PHARMACEUTICALS LLC, Edina, MN (US)

(72) Inventors: Yogesh DANDIKER, Edina, MN (US); Maulik K. PANCHAL, Maple Grove, MN (US)

(21) Appl. No.: 17/620,015

(22) PCT Filed: Jun. 25, 2020
(86) PCT No.: PCT/US2020/039517

§ 371 Date: Dec. 16, 2021

Related U.S. Application Data
(60) Provisional application No. 62/866,837, filed on Jun. 26, 2019.

(51) Int. Cl. A61K 31/138; A61K 9/00; A61K 9/16; A61K 9/20; A61K 9/24; A61K 9/50

(52) U.S. Cl. CPC A61K 31/138; A61K 9/0004; A61K 9/5078; A61K 9/2054; A61K 9/209; A61K 9/1676

(57) ABSTRACT

This disclosure provides pharmaceutical compositions comprising atomoxetine, a pharmaceutically acceptable salt of atomoxetine, or a combination thereof, that can be administered to a human subject in need thereof. The disclosure also provides pharmaceutical compositions for the treatment of orthostatic hypotension.

### FIELD OF DISCLOSURE

The present disclosure relates to pharmaceutical compositions of atomoxetine, a pharmaceutically acceptable salt of atomoxetine, or combinations thereof, methods of making the same, and methods of using the same for treating patients in need thereof.

### BACKGROUND

Orthostatic hypotension (OH), also known as postural hypotension, is a form of low blood pressure that occurs when a person stands up. The underlying causes of OH can be broadly divided into neurogenic and non-neurogenic categories. Neurogenic orthostatic hypotension (nOH) is a form of OH involving the nervous system, e.g., OH caused by a peripheral or central neurologic disorder. Such disorders can cause a deficiency or dysregulation of norepinephrine which is the primary neurotransmitter that regulates blood pressure in response to postural changes. Accordingly, one objective of OH treatment is to increase levels of norepinephrine in patients. One way to increase norepinephrine levels is to administer an agent that generates norepinephrine, for example, droxidopa. Alternatively, norepinephrine levels can be increased in patients by inhibiting the norepinephrine transporter which is responsible for norepinephrine reuptake, for example, atomoxetine.

Studies have shown that atomoxetine, a selective norepinephrine reuptake inhibitor, when given in a pediatric dose of 18 mg, significantly increases seated and standing systolic blood pressure compared to placebo. Atomoxetine requires tonic release of norepinephrine from the nerve terminals to elicit its pressor effect. Atomoxetine raises blood pressure in autonomic failure patients by increasing synaptic norepinephrine concentrations in postganglionic sympathetic neurons. Patients with central autonomic impairment (multiple system atrophy), who have intact postganglionic sympathetic fibers and residual sympathetic tone, have been shown to have large pressor responses to atomoxetine.

Thus, atomoxetine may be a promising agent for the symptomatic management of patients with primary OH in whom conventional therapies have failed.

Although atomoxetine has been approved for use in the treatment of Attention Deficit/Hyperactivity Disorder (ADHD), it has not been approved for use in the treatment of OH, and the pharmaceutical formulation approved for the treatment of ADHD may not be optimal to dose patients with OH. Therefore, there is a need for an improved dosage form for atomoxetine, which would be suitable for use in the treatment of OH.

### SUMMARY

The present invention is directed to oral dosage forms containing the active agent atomoxetine, or a pharmaceutically acceptable salt thereof, such as atomoxetine hydrochloride, that provide for sustained release of the active agent over several hours. These oral dosage forms may be formulated as an extended release dosage form, or as a dosage form that provides both an initial immediate release (or fast release) of a portion of the active agent in the dosage form, and an extended release (slower release) of the remainder of the active agent in the dosage form, such that the dosage form releases the active agent for at least 4 hours.

The dosage form of the invention will limit fluctuations in plasma drug concentration, providing a therapeutic effect that is more uniform over time than from an immediate release dosage form. To demonstrate the difference between an oral dosage form that does not provide sustained release of the active agent, and one that does, simulated plasma concentrations comparing twice daily administration of an atomoxetine immediate release dosage form to an atomoxetine extended release dosage form were generated are shown in FIG. 1, and in the following table:

TABLE A

| Time (hour) | IR 18 mg (BID) | ER 40 mg (OD) |
|---|---|---|
| 0 | 0 | 0 |
| 1 | 210 | 260 |
| 2 | 195 | 275 |
| 3 | 165 | 265 |
| 4 | 145 | 255 |
| 5 | 125 | 245 |
| 6 | 110 | 240 |
| 7 | 260 | 230 |
| 8 | 240 | 225 |
| 9 | 200 | 210 |
| 10 | 160 | 195 |
| 11 | 135 | 170 |
| 12 | 115 | 140 |
| 13 | 90 | 105 |
| 14 | 60 | 75 |
| 15 | 45 | 55 |
| 16 | 30 | 45 |
| 17 | 25 | 33 |
| 18 | 18 | 28 |
| 19 | 15 | 20 |
| 20 | 11 | 18 |
| 21 | 10 | 15 |
| 22 | 8 | 13 |
| 23 | 7 | 12 |
| 24 | 6 | 11 |

The oral dosage form may be, but is not limited to, a single or multi-layer (e.g., two, three or four layer) tablet, a capsule, a floating dosage form such as a floating tablet, an orally disintegrating tablet, a chewable tablet, a buccal adhesive tablet, a sublingual tablet, an oral suspension, or a powder, pellets, granules, or multi-particulates, which can be used, e.g., for an oral suspension. In some embodiments, the pharmaceutical composition is based on a dissolution-controlled release, diffusion-controlled release, diffusion- and dissolution-controlled release, ion-exchange release, pH-independent release, or an altered density formulation.

Certain aspects of the disclosure relate to a pharmaceutical composition comprising a sustained release agent and about 5 mg to about 110 mg of an active agent selected from the group consisting of atomoxetine, a pharmaceutically acceptable salt of atomoxetine, and a combination thereof, wherein the composition releases: at least about 30% of the total weight of the active agent by about 2 hours, at least about 30% but less than about 90% of the total weight of the active agent by about 4 hours, at least about 50% but less than about 95% of the total weight of the active agent by about 6 hours, and at least about 70% of the total weight of the active agent by about 8 hours, wherein the release profile is measured by an in vitro dissolution test. In some embodiments, the composition releases: at least about 40% of the total weight of the active agent by about 1 hour, at least about 40% but less than about 75% of the total weight of the active agent by about 2 hours, at least about 50% but less than about 80% of the total weight of the active agent by about 4 hours, and at least about 80% of the total weight of the active agent by about 8 hours, as measured by an in vitro dissolution test.

Certain aspects of the disclosure relate to a pharmaceutical composition comprising a sustained release agent and about 5 mg to about 110 mg of an active agent selected from the group consisting of atomoxetine, a pharmaceutically acceptable salt of atomoxetine, and a combination thereof, wherein the composition releases: at least about 20% of the total weight of the active agent by about 2 hours, at least about 25% but less than about 80% of the total weight of the active agent by about 4 hours, at least about 40% but less than about 90% of the total weight of the active agent by about 6 hours, and at least 90% of the total weight of the active agent by about 12 hours, as measured by an in vitro dissolution test. In some embodiments, this pharmaceutical composition releases: at least about 20% of the total weight of the active agent by about 1 hour, at least about 25% but less than about 60% of the total weight of the active agent by about 2 hours, at least about 30% but less than about 75% of the total weight of the active agent by about 4 hours, and at least about 80% of the total weight of the active agent by about 8 hours, as measured by an in vitro dissolution test.

In some embodiments, the in vitro dissolution test is performed with USP Apparatus I (baskets) at 100 rpm in 900 mL at 37° C., 0-2 hours, 0.1N HCl (pH 1.2); 2-4 hours, acetate buffer (pH 4.5); 4-12 hours, phosphate buffer (pH 6.8).

In some embodiments, the total amount of active agent in the composition is selected from the group consisting of about 10 mg, about 18 mg, about 40 mg, about 80 mg and about 100 mg.

In some embodiments, the active agent is atomoxetine HCl, and the total amount of active agent in the composition is equivalent to an amount of atomoxetine selected from the group consisting of about 10 mg, about 18 mg, about 40 mg, about 80 mg and about 100 mg.

In some embodiments, the active agent is present in an amount of about 4% to about 30% of the total weight of the composition.

In other embodiments, the active agent is present in an amount of about 2% to about 40% of the total weight of the composition, and the ratio of the amount of active agent to the amount of sustained release agent in the composition is about 1:1 to about 1:30 (w/w).

In yet other embodiments, the composition comprises a first release portion comprising about 20% to about 40% of the weight of the active agent in the composition; and a second release portion comprising about 40% to about 80% of the weight of the active agent in the composition, wherein the second release portion comprises a sustained release agent.

The composition may also comprise a third release portion that comprises a sustained release agent and an active agent selected from the group consisting of atomoxetine, a pharmaceutically acceptable salt of atomoxetine, and a combination thereof.

In some embodiments, the in vitro release rate of the active agent from the composition decreases within about 1 hour of the start of the in vitro dissolution test, and increases between about 1 to about 4 hours and subsequently increases between about 4 and about 8 hours after start of the in vitro dissolution test.

In yet other embodiments, the in vitro release rate of the active agent within the last 4 hours of release is slower than the in vitro release rate of the active agent released during the first 4 hours of release.

The invention further provides a pharmaceutical composition comprising: a first release portion and a second release portion, wherein the first release portion and the second release portion each comprise an active agent selected from the group consisting of atomoxetine, a pharmaceutically acceptable salt of atomoxetine, and a combination thereof; wherein the first release portion is an immediate release portion and the second release portion is an extended release portion and the second release portion releases the active agent at a slower rate than the release rate of the first release portion in an in vitro dissolution test; and wherein the composition releases: at least about 20% of the total weight of the active agent by about 2 hours, at least about 25% but less than about 70% of the total weight of the active agent by about 4 hours, at least about 40% but less than about 90% of the total weight of the active agent by about 6 hours, and at least 90% of the total weight of the active agent by about 12 hours, as measured by an in vitro dissolution test performed with USP Apparatus I (baskets) at 100 rpm in 900 mL at 37° C., 0-2 hours, 0.1N HCl (pH 1.2); 2-4 hours, acetate buffer (pH 4.5); 4-12 hours, phosphate buffer (pH 6.8)

In some embodiments, the active agent is present in the first release portion in an amount of about 20% to about 40% of the total weight of the active agent in the composition, and the active agent is present in the second release portion in an amount of about 40% to about 80% of the total weight of the active agent in the composition.

In some embodiments, the active agent in the first release portion and in the second release portion is atomoxetine HCl, the amount of active agent in the first release portion is equivalent to about 6 mg of atomoxetine, and the amount of active agent in the second release portion is equivalent to about 12 mg of atomoxetine.

The invention also provides for a method for treating or reducing the incidence of orthostatic hypotension (OH) in a subject in need thereof comprising administering the pharmaceutical composition to the subject.

In addition, it provides for a method for treating or reducing the incidence of postural orthostatic tachycardia syndrome (POTS) in a subject in need thereof comprising administering the pharmaceutical composition to the subject.

In some embodiments, the method comprises administering the pharmaceutical composition to a subject to treat or reduce the incidence of a condition selected from the group consisting of: orthostatic hypotension, postural orthostatic tachycardia syndrome (POTS), vasovagal syncope, dysautonomia, retrograde ejaculation or other disorder of semen ejaculation, symptoms of chronic orthostatic hypotension corresponding to autonomic failure associated with Bradbury-Eggleston, Shy-Drager syndromes, diabetes mellitus disease, and Parkinson's disease; in the subject.

The invention also provides a method for treating or reducing the incidence of vasovagal syncope in a subject in need thereof comprising administering the pharmaceutical composition to the subject.

In some embodiments, a unit dose of 18 mg of atomoxetine is administered once daily to the subject as an initial unit dose. In other embodiments, this subject may later be administered a higher unit dose once daily.

The invention also provides a kit comprising a first formulation and a second formulation, wherein the first formulation comprises a pharmaceutical composition of claim 1, 2, 3 or 4, and the second formulation comprises a second active agent. In some embodiments, the second active agent is selected from the group consisting of hydrocortisone, fludrocortisone, octreotide, pharmaceutically acceptable salts thereof, and a combination thereof.

In addition, the invention provides each of the embodiments of the pharmaceutical composition described above, for use in treating orthostatic hypotension in a subject in need thereof.

In another aspect, the invention provides each of the embodiments of the pharmaceutical composition described above, for use in reducing the incidence of orthostatic hypotension in a subject in need thereof.

In yet another aspect, the invention relates to each of the embodiments of the pharmaceutical composition described above, for use in treating a condition selected from the group consisting of: orthostatic hypotension, postural orthostatic tachycardia syndrome (POTS), vasovagal syncope, dysautonomia, retrograde ejaculation or other disorder of semen ejaculation, symptoms of chronic orthostatic hypotension corresponding to autonomic failure associated with Bradbury-Eggleston, Shy-Drager syndromes, diabetes mellitus disease, and Parkinson's disease; in a subject in need thereof.

In another aspect, the invention provides each of the embodiments of the pharmaceutical composition described above, for use in reducing the incidence of a condition selected from the group consisting of: orthostatic hypotension, postural orthostatic tachycardia syndrome (POTS), vasovagal syncope, dysautonomia, retrograde ejaculation or other disorder of semen ejaculation, symptoms of chronic orthostatic hypotension corresponding to autonomic failure associated with Bradbury-Eggleston, Shy-Drager syndromes, diabetes mellitus disease, and Parkinson's disease; in a subject in need thereof.

The invention also provides each of the embodiments of the use of the pharmaceutical compositions described above, wherein the active agent in the composition is atomoxetine HCl, and the total amount of active agent in the composition is equivalent to an amount of atomoxetine selected from the group consisting of about 10 mg, about 18 mg, about 40 mg, about 80 mg and about 100 mg.

### BRIEF DESCRIPTION OF THE DRAWINGS

FIG. 1 is a graph that depicts both (1) a simulated 24 hour plasma concentration (ng/mL) profile of atomoxetine for BID administration of an immediate capsule containing an amount of atomoxetine HCl equivalent to 18 mg atomoxetine (solid line); and (2) a simulated 24 hour plasma concentration (ng/mL) profile of atomoxetine for once daily administration of an extended release tablet containing an amount of atomoxetine HCl equivalent to 40 mg of atomoxetine.

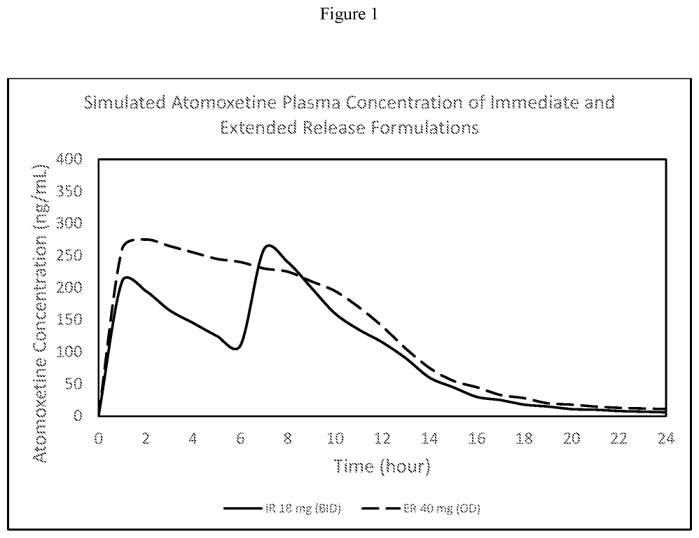

FIG. 2 is a graph that depicts the in vitro dissolution profile (shown as percentage of active agent released over a period of 12 hours) for a hydrophilic matrix tablet formulation containing an amount of atomoxetine HCl equivalent to 18 mg atomoxetine.

FIG. 3 is a graph that depicts in vitro dissolution profile (shown as amount (mg) of active agent released over a period of 12 hours) for a hydrophilic matrix tablet formulation containing an amount of atomoxetine HCl equivalent to 18 mg of atomoxetine.

FIG. 4 is a graph that depicts the in vitro dissolution profile (shown as percentage of active agent released over a period of 8 hours) for a bilayer tablet formulation containing an amount of atomoxetine HCl equivalent to 18 mg atomoxetine.

FIG. 5 is a graph that depicts the in vitro dissolution profile (shown as percent active agent released) for a bilayer tablet formulation containing an amount of atomoxetine equivalent to 18 mg atomoxetine, showing atomoxetine is released rapidly, followed by a sustained release of the drug, followed by a rapid release of the drug.

FIG. 6 is a graph that depicts in vitro dissolution profile (shown as amount (mg) of active agent released over a period of 12 hours) for a hydrophilic matrix tablet formulation containing an amount of atomoxetine HCl equivalent to 18 mg of atomoxetine, similar to that of Example 2.

### DETAILED DESCRIPTION

Certain aspects of the invention are directed to a pharmaceutical composition comprising the active agent atomoxetine, a pharmaceutically acceptable salt of atomoxetine (e.g., atomoxetine hydrochloride), or a combination thereof, wherein the composition provides for extended release of the active agent.

In some embodiments, the pharmaceutical composition includes different segments, referred to as different portions, which are each formulated to release the active agent at a different rate. For example, the pharmaceutical composition may include a first portion that is formulated with ingredients that allow immediate release of the active agent, and a second portion that is formulated with ingredients that provide extended release of the active agent (i.e., release that is slower than active agent release from the immediate release portion of the composition). Overall, the pharmaceutical composition sustains release of the active agent for at least four hours.

In some embodiments, the pharmaceutical composition contains only an extended release portion, in other embodiments it contains both an immediate release portion and an extended release portion. In yet other embodiments, the pharmaceutical may contain more than one immediate release portion with one extended release portion, or a single immediate release portion with multiple extended release portions, or multiple immediate release portions with multiple extended release portions. To distinguish the different segments—or portions from each other, they are referred to as a first portion, second portion, third portion, fourth portion, etc.

For example, the pharmaceutical composition may comprise a first portion that releases the active agent from that portion relatively quickly, e.g., releases at least 20% of the active agent in the composition within the first 1 hour after administration to a subject, and releases the second (or remaining) portion of the active agent over an extended period of time, e.g., over a period of at least 4 hours, 6 hours, 8 hours, 10 hours, 12 hours, 14 hours, or 16 hours, after administration to the subject. The pharmaceutical composition may further contain additional portions, such as a third portion that releases the active agent quickly, or a third portion that releases the active agent over an extended period.

In a preferred embodiment, the in vitro dissolution test conditions for evaluating drug release from the pharmaceutical composition are as follows: USP Apparatus I (baskets) at 100 rpm in 900 mL at 37° C., 0-2 hours, 0.1N HCl (pH 1.2); 2-4 hours, acetate buffer (pH 4.5); 4-16 hours (or 4-12 hours or 4-8 hours, depending on the length of the test), phosphate buffer (pH 6.8).

In some embodiments, the sustained release of atomoxetine, a pharmaceutically acceptable salt of atomoxetine, or a combination thereof, from the pharmaceutical composition of the invention maintains the blood pressure of the subject to which the composition is administered in a desired range throughout the day.

Thus, the pharmaceutical compositions of the invention may be used to treat or reduce the incidence of orthostatic hypotension (OH), or postural orthostatic tachycardia syndrome (POTS) in a subject.

In some embodiments, the subject suffers from orthostatic hypotension, dysautonomia, postural orthostatic tachycardia syndrome (POTS), retrograde ejaculation or other disorder of semen ejaculation, Bradbury-Eggleston, Shy-Drager syndromes, diabetes mellitus disease, or Parkinson's disease. In some embodiments, the subject suffers from orthostatic hypotension due to autonomic failure. In some embodiments, the subject suffers from vasovagal syncope.

Atomoxetine hydrochloride is well-absorbed after oral administration from immediate release capsules and is minimally affected by food. It has an absolute bioavailability of 63% in extensive metabolizers and 94% in poor metabolizers. Maximum plasma concentrations (Cₘₐₓ) are reached approximately 1 to 2 hours after dosing with an estimated half-life of about 5 hours.

Atomoxetine is metabolized primarily through the CYP2D6 enzymatic pathway. People with reduced activity in this pathway (poor metabolizers or PMs) have higher plasma concentrations of atomoxetine compared with people with normal activity (EMs). For PMs, AUC of atomoxetine is approximately 10-fold and Css, max is about 5-fold greater than EMs. The major oxidative metabolite formed, regardless of CYP2D6 status, is 4-hydroxyatomoxetine, which is glucuronidated. 4-Hydroxyatomoxetine is primarily formed by CYP2D6, but in PMs, it is formed at a slower rate by several other cytochrome P450 enzymes. N-Desmethylatomoxetine is formed by CYP2C19 and other cytochrome P450 enzymes, but has substantially less pharmacological activity compared with atomoxetine and circulates in plasma at lower concentrations. Mean apparent plasma clearance of atomoxetine after oral administration in adult EMs is 0.35 L/hr/kg and the mean half-life is 5.2 hours. Following oral administration of atomoxetine to PMs, mean apparent plasma clearance is 0.03 L/hr/kg and mean half-life is 21.6 hours. For PMs, AUC of atomoxetine is approximately 10-fold and Css, max is about 5-fold greater than EMs. The elimination half-life of 4-hydroxyatomoxetine is similar to that of N-desmethylatomoxetine (6 to 8 hours) in EM subjects, while the half-life of N-desmethylatomoxetine is much longer in PM subjects (34 to 40 hours).

Atomoxetine is excreted primarily as 4-hydroxyatomoxetine-O-glucuronide, mainly in the urine (greater than 80% of the dose) and to a lesser extent in the feces (less than 17% of the dose).

Patients suffering from OH report feeling worse in the morning, compared to afternoon and evening. Thus the inventors have discovered that the conventional immediate release capsule of atomoxetine hydrochloride is a poor dosage form for OH treatment as it creates a saw-tooth pattern of high and low concentrations of atomoxetine in the plasma, and would require more than once daily dosing.

Another disadvantage of using an immediate release formulation to treat OH is that such a formulation would need to be administered more than once per day, yet would still not sufficiently maintain a patient's blood pressure for periods sufficient to manage symptoms such as dizziness and/or light headedness throughout the day. Multiple doses of immediate release atomoxetine could result in repeated rises and falls in the patient's blood pressure as the active agent is released upon each administration and subsequently eliminated. Therefore, in some embodiments of the invention, the formulations disclosed herein can maintain the patient's blood pressure throughout the day and thereby reduce the occurrence of symptoms such as dizziness and/or light headedness.

In addition, often the blood pressure of OH patients is lowest in the morning, and some of the patients' symptoms are worst in the morning. Because the pharmaceutical compositions of the invention provide both immediate (fast) release of the active agent followed by sustained release of the active agent, if administered in the morning, the immediate release of active agent can provide relief for these symptoms when most needed, whereas extended release dosage forms that do not allow for an initial immediate release of the active agent would not address this need.

Moreover, the multiple dosing of immediate release oral dosage forms decreases patient compliance. Missing or delayed administration of an immediate release dose can result in the patient's condition becoming worse and sometimes unmanageable. The present application provides a formulation of the drug that can be given less frequently than the immediate release oral dosage forms, and thereby improve patient compliance.

In addition, the currently available formulation of atomoxetine is a capsule, and many patients find difficulty in swallowing capsules.

### Definitions

As used herein, the following words and phrases are generally intended to have the meanings as set forth below, except to the extent that the context in which they are used indicates otherwise.

As used in the specification and the appended claims, the singular forms “a,” “an,” and “the” include plural reference unless the context clearly dictates otherwise.

The term “about” as used herein means approximately ±10%. When the term “about” is used in conjunction with a numerical value or range, it modifies that value or range by extending the boundaries above and below the numerical values set forth. In general, the term “about” is used herein to modify a numerical value above and below the stated value by a variance of 10 percent, up or down (higher or lower), i.e., ±10%, unless a different variance is indicated (e.g., ±30%, ±20%, ±5%, ±1%, etc.).

Also as used herein, “and/or” refers to and encompasses any and all possible combinations of one or more of the associated listed items, as well as the lack of combinations when interpreted in the alternative (“or”).

Furthermore, when the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See Bryan A. Garner, A Dictionary of Modern Legal Usage 624 (2d. Ed. 1995). Also, to the extent that the terms “in” or “into” are used in the specification or the claims, it is intended to additionally mean “on” or “onto.”

The term “active agent” refers to a substance, including a biologically active substance, that is useful for prophylactic and/or therapeutic treatment. Typically, the active agents are organic molecules that are drug compounds, their salts, metabolites, etc. The term “active agent” as used in this disclosure can refer to atomoxetine, a pharmaceutically acceptable salt of atomoxetine, or a combination thereof. For example, the active agent can be atomoxetine hydrochloride (HCl).

The term “atomoxetine” refers to the compound with the chemical designation (−)-N-Methyl-3-phenyl-3-(o-tolyloxy)-propylamine, having the following chemical structure:

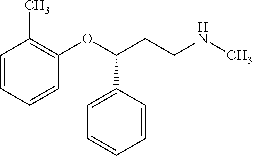 [SMILES: CNCC[C@@H](Oc1ccccc1C)c1ccccc1]

Atomoxetine HCl is the R(−) isomer as determined by x-ray diffraction. The chemical designation is (−)-N-Methyl-3-phenyl-3-(o-tolyloxy)-propylamine hydrochloride. The molecular formula is C17H21NO.HCl, which corresponds to a molecular weight of 291.82. The chemical structure is:

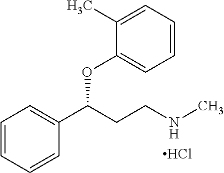 [SMILES: CNCC[C@@H](Oc1ccccc1C)c1ccccc1 ; Cl]

For purposes of this disclosure, unless indicated otherwise, when referring to a formulation or pharmaceutical composition comprising “atomoxetine”, it should be understood that the embodiment can include atomoxetine or a pharmaceutically acceptable salt of atomoxetine, e.g., atomoxetine HCl. Unless otherwise specified, the doses described in this application (e.g., 10, 18, 40, 80, 100 mg) refer to the weight of atomoxetine in the dosage form, rather than the weight of atomoxetine HCl in the dosage form. Thus, the dosage forms described in the Examples contain atomoxetine HCl in an amount equivalent to the stated amount of atomoxetine in the Tables in those Examples. For example, if the desired dose of atomoxetine in the unit dosage form is 18 mg, the weight of atomoxetine HCl in the unit dosage form would be about 20.57 mg.

The term “pharmaceutically acceptable salt” of a given compound refers to salts that retain the biological effectiveness and properties of the given compound, and which are not biologically or otherwise undesirable. “Pharmaceutically acceptable salts” include, for example, salts with inorganic acids and salts with an organic acid. Compounds described herein as an acid addition salt may be formed into a corresponding the free base by basifying a solution of the acid salt. Conversely, an addition salt, particularly a pharmaceutically acceptable addition salt, may be produced by dissolving a corresponding free base in a suitable organic solvent and treating the solution with an acid, in accordance with conventional procedures for preparing acid addition salts from base compounds.

Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, sodium, potassium, lithium, ammonium, calcium and magnesium salts. Salts derived from organic bases include, but are not limited to, salts of primary, secondary and tertiary amines. In one embodiment, a pharmaceutically acceptable salt of atomoxetine is a hydrochloride salt.

The term “portion” as used herein refers to a part, a segment, or a share of a whole or total (e.g., of a whole tablet or a total amount of active agent).

The term “substantially all” as used herein refers to most of the total amount, e.g., at least 80%, at least 85%, at least 90%, at least 95% of a total amount.

The term “subject,” as used herein, refers to a human, e.g., a human patient.

The term “first release portion”, “fast release portion” and “immediate release portion” as used herein refer to a part of the pharmaceutical composition disclosed herein that releases substantially all of the active agent contained in that portion relatively quickly, e.g., within about 30 minutes, about 1 hour, or about 1.5 hours, after the start of an in vitro dissolution test. In some embodiments, the first release portion comprises an immediate release delivery system.

The terms “immediate release” and “fast release” as used herein refer to a delivery system in which at least a portion of the active agent is released from a pharmaceutical composition or formulation without delay. In some embodiments, substantially all of the immediate release of the active agent is within about 30 minutes, about 1 hour, or about 1.5 hours, after the start of an in vitro dissolution test.

The term “second release portion” or “extended release portion” as used herein refers to a part of the pharmaceutical composition disclosed herein that sustains release of the active agent contained in that portion over a time period longer than an hour, e.g., for a period of at least 4 hours, after the start of an in vitro dissolution test. In some embodiments, the second release portion comprises an extended release delivery system.

The term “extended release” as used herein refers to a delivery system by which the release of the active agent is sustained over a time period (i.e., an extended release period). In some embodiments, the pharmaceutical composition provides release of the active agent over a period of up to about 4 hours, up to about 6 hours, up to about 8 hours, up to about 10 hours, up to about 12 hours, up to about 14 hours, about 16 hours, after the start of an in vitro dissolution test.

The in vitro dissolution conditions for the immediate release/extended release compositions disclosed herein are designed to mimic the in vivo release conditions. In a preferred embodiment, the in vitro dissolution test conditions are as follows: USP Apparatus I (baskets) at 100 rpm in 900 mL at 37° C., 0-2 hours, 0.1N HCl (pH 1.2); 2-4 hours, acetate buffer (pH 4.5); 4-16 hours (or 4-12 hours or 4-8 hours, depending on the length of the test), phosphate buffer (pH 6.8).

The term “rate controlling agent” refers to an agent whose primary function is to modify the duration of release of the active drug substance from a dosage form. In preferred embodiments, the rate controlling agent is a sustained release agent, which is an agent that provides for slower release of the active agent than if the agent were not present in the formulation. In some embodiments, the rate controlling agent is a fast release agent, which is an agent that speeds release of the active agent from the formulation.

“Percent” or “%” as used herein expresses the number of parts per 100. Where percent or % is used with regard to concentration or weight, it refers to the number of parts of the particular ingredient in a total of 100 parts weight or volume. For example, a product expressed as 0.3% w/v means 0.3 gm in 100 mL. A product expressed as 10% w/w is interpreted as 10 gm in 100 gm. A 0.05% w/v API solution means that 100 ml of the product contains 0.05 g of API. In another example, 0.1% w/w API means there is 0.1 gm of API in every 100 gm of product.

The term “percent drug released” as used herein refers to the percentage of the active agent released from a pharmaceutical composition at a specified time as compared to the total amount of the active agent in the pharmaceutical composition. For example, if a pharmaceutical composition releases 20% of the total weight of the active agent at by 1 hour, 28% of the total weight of the active agent by 2 hours, and 35% of the total weight of the active agent by 3 hours (cumulative) in a dissolution test, then the percent drug released of this pharmaceutical composition is 20% by 1 hour, 28% by 2 hours, and 35% by 3 hours (cumulative).

The term “release rate” refers to the amount of active agent released from the pharmaceutical composition over a period of time. For example, the release rate can be reported as the amount of active agent released per hour (e.g., mg/hr) or the percentage of the active agent released per hour (e.g., %/hr). These amounts reflect only the amount released since the last timepoint at which release was measured. The release rate can be measure with an in vitro test, such as an in vitro dissolution test, or an in vivo test. As described herein, the drug release rate can be measured approximately by incremental increases in the percent drug released at a specified time point as compared to the preceding time point in an in vitro dissolution test. For example, if the percent drug released of a pharmaceutical composition is 20% by 1 hour, 28% by 2 hours, and 35% by 3 hours (cumulative), the drug release rate can be described as 20%/hr at 1 hour, 8%/hr at 2 hours, and 7%/hr at 3 hours.

The term “release profile” refers to an in vitro dissolution curve representing the amount or percentage of active agent measured at specific time points. The term “release profile” can also refer to an in vivo plasma curve representing the active agent plasma concentration measured at specific time points.

The term “rise” as used herein refers to an increase of the active agent release rate from a composition to a peak (or shoulder or plateau) in its in vitro dissolution release profile and/or the term “rise” can also refer to an increase of the active agent plasma concentration to a peak (or shoulder or plateau) in its in vivo release profile. As discussed above, the active agent release rate in an in vitro dissolution test can be approximately described by the incremental increase in the percent active agent released at a specified time point as compared to the preceding time point in the dissolution test.

The term “second rise” as used herein refers to an increase of the active agent release rate from a composition after the initial release or first rise. In some embodiments, the disclosed formulations do not have a second rise taking place 5 hours (e.g., between about 5-10 hours) after start of an in vitro dissolution test.

The term “third rise” as used herein refers to an increase of the active agent release rate from a composition after the initial release (or first rise) and after the second rise.

The term “treatment” or “treating” means any treatment of a disease in a mammal, including:
(i) preventing or reducing the incidence of the disease by causing the clinical symptoms of the disease not to develop;
(ii) inhibiting the disease by arresting the development of clinical symptoms of the disease; and/or
(iii) relieving the disease by causing the regression of clinical symptoms of the disease.

An “effective amount” is the quantity of compound or formulation in which a beneficial clinical outcome can be achieved when the compound or formulation is administered to a subject suffering from or at risk of suffering from a condition to be treated. A “beneficial clinical outcome” can include one or more of: a reduction in number or severity of symptoms in a subject, such as an increase in blood pressure, lack of dizziness and/or lack of light headedness.

As used herein, “multi-particulates” (used interchangeably with “a multi-particulate”) refers to discrete, small drug units, exhibiting a desired characteristic, that make up a multiple unit drug delivery system. The multi-particulates can be in the form of, for example, a drug particle, a granule, a pellet, a bead, a sphere, or a mini-tablet.

Wherever aspects or features are described herein with the language “comprising,” otherwise analogous aspects or features described in terms of “consisting of” and/or “consisting essentially of” are also provided. To the extent that the term “includes” or “including” is used in the specification or the claims, it is intended to be inclusive in a manner similar to the term “comprising” as that term is interpreted when employed as a transitional word in a claim.

As used herein, the terms “optional” and “optionally” mean that the subsequently described circumstance may or may not occur, so that the description includes instances where the circumstance occurs and instances where it does not.

Where features or aspects of the disclosure or claims are described in terms of Markush groups, those skilled in the art will recognize that the disclosure is also thereby described in terms of any individual member or subgroup of members of the Markush group.

In addition, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, and the like. As a non-limiting example, each range discussed herein can be readily broken down into a lower third, middle third and upper third, and the like. As will also be understood by one skilled in the art all language such as “up to,” “at least,” “greater than,” “less than,” include the number recited and refer to ranges which can be subsequently broken down into sub-ranges as discussed above. Finally, as will be understood by one skilled in the art, a range includes each individual member. For example, a group having 1-3 members refers to groups having 1, 2, or 3 members. Similarly, a group having 1-5 members refers to groups having 1, 2, 3, 4, or 5 members, and so forth.

While various aspects and embodiments have been disclosed herein, other aspects and embodiments will be apparent to those skilled in the art.

### Pharmaceutical Compositions

In some embodiments, the pharmaceutical compositions disclosed herein can contain one or more active agents. The active agents can be selected from the group consisting of atomoxetine, a pharmaceutically acceptable salt of atomoxetine, and a combination thereof. In one embodiment, the active agent is atomoxetine or a pharmaceutically acceptable salt thereof, e.g., atomoxetine hydrochloride (HCl).

In some embodiments, the pharmaceutical compositions disclosed herein can be, but are not limited to, a single or multi-layer tablet, a capsule, a floating dosage form such as a floating tablet, an orally disintegrating tablet, a chewable tablet, a buccal adhesive tablet, a sublingual tablet, an oral suspension, multi-particulates, an oral suspension, such as a suspension containing multi-particulates.

In some embodiments, the pharmaceutical composition is based on a dissolution-controlled release, diffusion-controlled release, diffusion- and dissolution-controlled release, ion-exchange release, pH-independent release, or an altered density formulation.

In some embodiments, the pharmaceutical compositions envisioned herein include, but are not limited to:

1. a matrix extended release tablet;

2. a bi-layer tablet containing an immediate release layer and an extended release layer;

3. a tri-layer tablet containing an immediate release layer and two extended release layers, or an extended release layer and two immediate release layers;

4. an immediate release drug coating on an extended release core tablet;

5. a coated tablet having an extended release core and immediate release coating;

6. an immediate release core coated with extended release coating followed by a further immediate release drug coating;

7. a tablet-in-tablet dosage form that includes an extended release tablet in an immediate release tablet;

8. a capsule filled with multi-particulates (e.g., pellets, particles, granules, beads, spheres, or mini-tablets) or a tablet compressed from multi-particulates in which the multi-particulates have an extended release core and immediate release coating;

9. a blend of immediate release and extended release multi-particulates that may be filled within a capsule or compressed into a tablet;

10. a blend of immediate release and extended release multi-particulates that may be suspended in an appropriate delivery vehicle known in the art or filled into a sachet for reconstitution;

11. an oral suspension containing extended release multi-particulates, or both extended release multi-particulates and immediate release multi-particulates;

12. a floating dosage form, such as a floating tablet, or floating pellets;

13. an extended release osmotic controlled tablet with immediate release drug layer coat.

In some embodiments, the extended release portion of the pharmaceutical composition, comprises one or more sustained release agents.

In some embodiments, the total weight of the active agent in the pharmaceutical composition is about 5 mg to about 150 mg, about 5 mg to about 110 mg, about 7.5 mg to about 150 mg, about 7.5 mg to about 120 mg, about 7.5 mg to about 100 mg, about 7.5 mg to about 75 mg, about 7.5 mg to about 50 mg, about 10 mg to about 120 mg, about 10 mg to about 100 mg, about 10 mg to about 80 mg, about 10 mg to about 40 mg, about 10 mg to about 20 mg, about 10 mg to about 18 mg, about 18 mg to about 100 mg, about 18 mg to about 80 mg, about 18 mg to about 40 mg, about 40 mg to about 100 mg, about 40 mg to about 80 mg, about 80 to about 100 mg, about 20 mg to about 75 mg, about 20 mg to about 50 mg, about 25 mg to about 35 mg, or about 40 mg to about 50 mg.

In some embodiments, the total weight of the active agent in the pharmaceutical composition is about 5 mg, about 5.5 mg, about 6 mg, about 6.5 mg, about 7 mg, about 7.5 mg, about 8 mg, about 8.5 mg, about 9 mg, about 9.5 mg, about 10 mg, about 12.5 mg, about 15 mg, about 17.5 mg, about 18 mg, about 20 mg, about 20.5 mg, about 22.5 mg, about 25 mg, about 27.5 mg, about 30 mg, about 32.5 mg, about 35 mg, about 37.5 mg, about 38 mg, about 40 mg, about 41 mg, about 42 mg, about 42.5 mg, about 45 mg, about 47.5 mg, about 48 mg, about 50 mg, about 52.5 mg, about 55 mg, about 57.5 mg, about 60 mg, about 62.5 mg, about 65 mg, about 67.5 mg, about 70 mg, about 72.5 mg, about 75 mg, about 77.5 mg, about 80 mg, about 90 mg, about 100 mg, about 110 mg, about 120 mg, about 130 mg, about 140 mg, or about 150 mg.

In some embodiments, the total weight of the active agent in the pharmaceutical composition is about 10 mg, about 18 mg, about 40 mg, about 80 mg, or about 100 mg of atomoxetine, or is an amount of atomoxetine HCl that is equivalent to one of these doses of atomoxetine.

In some embodiments, the active agent is present in an amount of about 2% to about 40%, about 4% to about 30%, about 4% to about 12%, about 10% to about 40%, or about 10% to about 30%, of the total weight of the composition.

In some embodiments, the pharmaceutical compositions disclosed herein can comprise multiple distinct and distinguishable portions which can comprise the same or different excipients. In one embodiment, the pharmaceutical compositions can comprise an immediate release portion and an extended release portion (e.g., wherein the immediate release and extended release portions each comprise atomoxetine hydrochloride, but where the portions differ with regard to whether they contain a sustained release agent). In other embodiments, the pharmaceutical compositions can comprise an immediate release portion, and multiple extended release portions, and wherein the composition of each of these portions differs from each other.

In other embodiments, the pharmaceutical composition does not contain separate immediate release and extended portions. Instead, the pharmaceutical composition may contain only an extended release portion (a matrix extended release formulation), although such a formulation may have one or more coatings that do not contain active agent or rate controlling agent, such as a film coating.

In some embodiments, the immediate release portion and extended release portion of the pharmaceutical composition each comprise atomoxetine hydrochloride. In some embodiments, the matrix extended release formulation comprises atomoxetine hydrochloride.

In some embodiments, the immediate release portion comprises about 10% to about 50%, about 15% to about 45%, about 20% to about 40%, about 30% to about 40%, about 30% to about 50%, or about 30% to about 35% of the total weight of the active agent in the composition.

In another embodiment, the immediate release portion can comprise an active agent in an amount of about 15%, about 20%, about 21%, about 22%, about 23%, about 24%, about 25%, about 26%, about 27%, about 28%, about 29%, about 30%, about 31%, about 32%, about 33%, about 34%, about 35%, about 40%, about 45%, or about 50% of the total weight of the active agent in the composition.

In some embodiments, the extended release portion comprises an active agent in an amount of about 50% to about 90%, about 50% to about 80%, about 55% to about 75%, about 60% to about 80%, about 60% to about 75%, about 60% to about 70%, or about 65% to about 70% of the total weight of the active agent in the composition.

In another embodiment, the extended release portion can comprise an active agent in an amount of about 50%, about 55%, about 60%, about 65%, about 70%, about 75%, about 80%, or about 85% w/w of the total weight of the active agent in the composition.

In yet other embodiments, the composition comprises a first release portion comprising about 20% to about 40% of the weight of the active agent in the composition; and a second release portion comprising about 60% to about 80% of the weight of the active agent in the composition, wherein the second release portion comprises a sustained release agent.

In some embodiments, the active agent is present in the first release portion in an amount of about 20% to about 40% of the total weight of the active agent in the composition, and the active agent is present in the second release portion in an amount of about 60% to about 80% of the total weight of the active agent in the composition.

In some embodiments, the immediate release portion comprises about 1 mg to about 40 mg of atomoxetine hydrochloride, and the extended release portion comprises about 10 mg to about 100 mg of atomoxetine hydrochloride. In some embodiments, the immediate release portion and the extended release portion comprise about 6 mg and about 12 mg of atomoxetine hydrochloride, respectively.

The extended release portion, or matrix extended release formulation, of the pharmaceutical compositions disclosed herein can comprise an effective amount of one or more sustained release agents. The sustained release agents can be, for example, water-soluble, water-insoluble, water permeable, water-impermeable excipients, and mixtures thereof. The sustained release agents can be a polymer or a non-polymeric agent. The sustained release agents can be a hydrophilic polymer or a hydrophobic polymer. In one embodiment, the sustained release agent is incorporated into the extended release portion. In another embodiment, the extended release portion of the pharmaceutical composition is coated with the sustained release agent. In yet another embodiment, the sustained release agent is found in all portions of the composition containing the active agent.

In some embodiments, the sustained release agent is a hydrophilic polymer selected from hypromellose; hydroxypropyl cellulose (HPC); hydroxyethyl cellulose (HEC); polyethylene oxide; polyvinyl alcohol; povidone; xanthan gum; guar gum; chitosan; a chitosan derivative; carbomer; carrageenan; carboxymethyl cellulose; sodium alginate; a polyglycolized glyceride; polyethylene glycol; a polyvinyl acetate dispersion; cellulose acetate; cellulose acetate butyrate; cellulose acetate phthalate; cellulose triacetate; and a combination thereof. In one embodiment the sustained release agent is hypromellose. In another embodiment, the sustained release agent is a hypromellose (e.g., USP hypromellose 2208) or METHOCEL™ K4M (COLORCON®).

In other embodiments, the sustained release agent is a hydrophobic polymer selected from poly(methyl methacrylate); poly(ethyl methacrylate); poly(methyl acrylate); poly(isopropyl acrylate); poly(isobutyl acrylate); poly(octadecyl acrylate); ethyl cellulose; cellulose propionate; cellulose acetate propionate; and a combination thereof.

In some embodiments, the sustained release agent is a non-polymeric agent selected from a wax; a fatty alcohol; a fatty acid ester; hydrogenated vegetable oil; and a combination thereof.

In some embodiments, the ratio of active agent to sustained release agent in the extended release portion, or in the matrix extended release formulation, of the composition is about 1:1 to about 1:30 (w/w), about 1:1 to about 1:20 (w/w), about 1:2 to about 1:20 (w/w), about 1:2 to about 1:15 (w/w), about 1:3 to about 1:20 (w/w), about 1:3 to about 1:15 (w/w), about 1:5 to about 1:20 (w/w), about 1:5 to about 1:15 (w/w), or about 1:5 to about 1:10 (w/w). In other embodiments, the ratio of active agent to sustained release agent in the extended release portion, or in the matrix extended release formulation, of the composition is about 1:5 (w/w), about 1:7 (w/w), about 1:10 (w/w), about 1:12 (w/w), about 1:15 (w/w), about 1:20 (w/w), about 1:25 (w/w), or about 1:30 (w/w).

The pharmaceutical compositions disclosed herein can comprise one or more fillers, binders, diluents, disintegrants, surfactants, pigments, lubricants, glidants, flavoring agents, pH adjusting agents, solubilizing agents, wetting agents, buffering agents, or any combination thereof.

Examples of binders include, but are not limited to, acacia, alginic acid, carbomer, carboxymethylcellulose sodium, dextrin, ethylcellulose, gelatin, guar gum, hydrogenated vegetable oil (type I), hydroxyethyl cellulose, hydroxypropyl cellulose, hydroxypropyl methylcellulose, liquid glucose, magnesium aluminum silicate, maltodextrin, methylcellulose, microcrystalline cellulose, polymethacrylates, povidone, pregelatinized starch, sodium alginate, starch, zein, and the like, or mixtures thereof. In one embodiment, the binder is hydroxypropyl methyl cellulose, microcrystalline cellulose, or povidone.

Examples of diluents include, but are not limited to, microcrystalline cellulose, lactose, starch, sucrose, calcium phosphate anhydrous and dibasic, mannitol, sorbitol, xylitol, maltitol, ammonium alginate, calcium carbonate, calcium lactate, calcium silicate, calcium sulfate, cellulose powdered, silicified microcrystalline cellulose, compressible sugar, confectioner's sugar, corn starch, pregelatinized starch, dextrates, dextrin, dextrose, erythritol, ethyl cellulose, fructose, glyceryl palmitosterate, isomalt, lactitol monohydrate, magnesium carbonate, magnesium oxide, maltose, medium chain triglycerides, polydextrose, polymethacrylates, simethicone, sodium alginate, sodium chloride, sterilize maize and a combination thereof.

Examples of disintegrants include, but are not limited to, alginic acid, carboxymethylcellulose calcium, carboxymethylcellulose sodium, colloidal silicon dioxide, croscarmellose sodium, crospovidone, guar gum, magnesium aluminum silicate, methylcellulose, microcrystalline cellulose, polyacrilin potassium, powdered cellulose, pregelatinized starch, sodium alginate, calcium alginate, powdered cellulose, glycine, sodium starch glycolate, starch, hydroxypropyl cellulose, and the like, or mixtures thereof.

Examples of glidants include, but are not limited to, starch, talc, silicon dioxide, e.g., colloidal silicon dioxide, and the like, or mixtures thereof.

Examples of lubricants include, but are not limited to, magnesium stearate, stearic acid, talc, calcium stearate, glyceryl monostearate, glyceryl behenate, glyceryl palmitostearate, hydrogenated castor oil, hydrogenated vegetable oil, mineral oil, polyethylene glycol, sodium benzoate, sodium lauryl sulfate, magnesium lauryl sulfate, medium chain triglycerides, myristic acid, palmitic acid, poloxamer, sodium benzoate, sodium stearyl fumarate, zinc stearate, and the like, or mixtures thereof.

In some embodiments, the pharmaceutical compositions disclosed herein can be further coated with a film coating such that the film coating does not modify the release characteristics of the composition. Suitable materials that can be used to film-coat the compositions include, but are not limited to, hypromellose, hydroxy propyl cellulose, polyvinyl alcohol, ready-to-use premix like Opadry® (hypromellose, PEG) (Colorcon), Opadry® II (polyvinyl alcohol, PEG, talc, and titanium dioxide), and mixtures thereof.

Certain aspects of the disclosure relate to a pharmaceutical composition comprising a sustained release agent and about 5 mg to about 110 mg of an active agent selected from the group consisting of atomoxetine, a pharmaceutically acceptable salt of atomoxetine, and a combination thereof, wherein the composition releases: at least about 30% of the total weight of the active agent by about 2 hours, at least about 30% but less than about 90% of the total weight of the active agent by about 4 hours, at least about 50% but less than about 95% of the total weight of the active agent by about 6 hours, and at least about 70% of the total weight of the active agent by about 8 hours, wherein the release profile is measured by an in vitro dissolution test. In some embodiments, the composition releases: at least about 40% of the total weight of the active agent by about 1 hour, at least about 40% but less than about 75% of the total weight of the active agent by about 2 hours, at least about 50% but less than about 80% of the total weight of the active agent by about 4 hours, and at least about 80% of the total weight of the active agent by about 8 hours, as measured by an in vitro dissolution test.

Certain aspects of the disclosure relate to a pharmaceutical composition comprising a sustained release agent and about 5 mg to about 110 mg of an active agent selected from the group consisting of atomoxetine, a pharmaceutically acceptable salt of atomoxetine, and a combination thereof, wherein the composition releases: at least about 20% of the total weight of the active agent by about 2 hours, at least about 25% but less than about 80% of the total weight of the active agent by about 4 hours, at least about 40% but less than about 90% of the total weight of the active agent by about 6 hours, and at least 90% of the total weight of the active agent by about 12 hours, as measured by an in vitro dissolution test. In some embodiments, this pharmaceutical composition releases: at least about 20% of the total weight of the active agent by about 1 hour, at least about 25% but less than about 60% of the total weight of the active agent by about 2 hours, at least about 30% but less than about 75% of the total weight of the active agent by about 4 hours, and at least about 80% of the total weight of the active agent by about 8 hours, as measured by an in vitro dissolution test.

In some embodiments, the in vitro dissolution test is performed with USP Apparatus I (baskets) at 100 rpm in 900 mL at 37° C., 0-2 hours, 0.1N HCl (pH 1.2); 2-4 hours, acetate buffer (pH 4.5); 4-12 hours, phosphate buffer (pH 6.8).

In some embodiments, the in vitro release rate of the active agent from the composition decreases within about 1 hour of the start of the in vitro dissolution test, and increases between about 1 to about 4 hours and subsequently increases between about 4 and about 8 hours after start of the in vitro dissolution test.

In yet other embodiments, the in vitro release rate of the active agent within the last 4 hours of release is slower than the in vitro release rate of the active agent released during the first 4 hours of release.

In some embodiments the pharmaceutical composition is characterized by the following release profile:
at least about 10%, about 15%, about 20%, about 25%, about 30%, about 35%, about 40%, about 45%, or about 50%, of the total weight of the active agent is released by about 30 minutes, about 1 hour, about 1.5 hours, or about 2 hours;
at least about 20% but less than about 90%, at least about 20% but less than about 85%, at least about 20% but less than about 80%, at least about 20% but less than about 75%, at least about 20% but less than about 70%, at least about 20% but less than about 65%, at least about 30% but less than about 90%, at least about 30% but less than about 85%, at least about 30% but less than 80%, at least about 30% but less than about 75%, at least about 30% but less than about 70%, at least about 30% but less than about 65%, at least about 30% but less than about 60%, at least about 35% but less than about 90%, at least about 35% but less than about 85%, at least about 35% but less than about 80%, at least about 35% but less than about 75%, at least about 35% but less than about 70%, at least about 35% but less than about 65%, at least about 35% but less than about 60%, at least about 35% but less than about 55%, at least about 40% but less than about 95%, at least about 40% but less than about 90%, at least about 40% but less than about 85%, at least about 40% but less than about 80%, at least about 40% but less than about 75%, at least about 40% but less than about 70%, at least about 40% but less than about 65%, at least about 40% but less than about 60%, at least about 40% but less than about 55%, at least about 50% but less than about 90%, at least about 50% but less than about 85%, at least about 50% but less than about 80%, at least about 50% but less than about 75%, at least about 60% but less than about 90%, at least about 60% but less than about 85%, at least about 60% but less than about 80%, at least about 60% but less than about 75%, at least about 65% but less than about 90%, at least about 65% but less than about 85%, at least about 65% but less than about 80%, or at least about 65% but less than about 75%, of the total weight of the active agent is released by about 4 hours;
at least about 30% but less than about 95%, at least about 30% but less than about 90%, at least about 30% but less than about 85%, at least about 30% but less than 80%, at least about 30% but less than about 75%, at least about 30% but less than about 70%, at least about 30% but less than about 65%, at least about 30% but less than about 60%, at least about 35% but less than about 95%, at least about 35% but less than about 90%, at least about 35% but less than about 85%, at least about 35% but less than about 80%, at least about 35% but less than about 75%, at least about 35% but less than about 70%, at least about 35% but less than about 65%, at least about 35% but less than about 60%, at least about 35% but less than about 55%, at least about 40% but less than about 95%, at least about 40% but less than about 90%, at least about 40% but less than about 85%, at least about 40% but less than about 80%, at least about 40% but less than about 75%, at least about 40% but less than about 70%, at least about 40% but less than about 65%, at least about 40% but less than about 60%, at least about 40% but less than about 55%, at least about 50% but less than about 95%, at least about 50% but less than about 90%, at least about 50% but less than about 85%, at least about 50% but less than about 80%, at least about 50% but less than about 75%, at least about 50% but less than about 70%, at least about 50% but less than about 65%, at least about 50% but less than about 60%, at least about 50% but less than about 55%, at least about 55% but less than about 95%, at least about 55% but less than about 90%, at least about 55% but less than about 85%, at least about 55% but less than about 80%, at least about 55% but less than about 75%, at least about 55% but less than about 70%, at least about 55% but less than about 65%, at least about 55% but less than about 60%, at least about 65% but less than about 95%, at least about 65% but less than about 90%, at least about 65% but less than about 85%, at least about 65% but less than about 80%, at least about 65% but less than about 75%, or at least about 65% but less than about 70%, of the total weight of the active agent is released by about 6 hours;
at least about 40% but less than about 100%, at least about 40% but less than about 95%, at least about 40%, but less than about 90%, at least about 40% but less than about 85%, at least about 40%, but less than about 80%, at least about 40% but less than about 75%, at least about 45% but less than about 100%, at least about 45% but less than about 95%, at least about 45% but less than about 90%, at least about 45% but less than about 85%, at least about 45% but less than about 80%, at least about 45% but less than about 75%, at least about 50% but less than about 100%, at least about 50% but less than about 95%, at least about 50% but less than about 90%, at least about 50% but less than about 85%, at least about 50% but less than about 80%, at least about 50% but less than about 75%, at least about 50% but less than about 70%, at least about 50% but less than about 65%, at least about 55% but less than about 100%, at least about 55% but less than about 95%, at least about 55% but less than about 90%, at least about 55% but less than about 85%, at least about 55% but less than about 80%, at least about 55% but less than about 75%, at least about 55% but less than about 70%, at least about 65% but less than about 100%, at least about 65% but less than about 95%, at least about 65% but less than about 90%, at least about 65% but less than about 85%, at least about 65% but less than about 80%, at least about 65% but less than about 75%, or at least about 65% but less than about 70%, at least about 70% but less than about 100%, at least about 70% but less than about 95%, at least about 70% but less than about 90%, at least about 70% but less than about 85%, at least about 70% but less than about 80%, at least about 70% but less than about 75%, at least about 75% but less than about 100%, at least about 75% but less than about 95%, at least about 75% but less than about 90%, at least about 75% but less than about 85%, at least about 75% but less than about 80%, at least about 80% but less than about 100%, at least about 80% but less than about 95%, at least about 80% but less than about 90%, at least about 80% but less than about 85%, or at least about 80%, at least about 85%, at least about 90% or at least about 95% of the total weight of the active agent is released by about 8 hours;
and at least about 80%, at least about 85%, at least about 90%, at least about 95% or at least about 98% of the total weight of the active agent is released by about 10 hours, about 12 hours, about 14 hours, or about 16 hours;
as measured by an in vitro dissolution test performed with USP Apparatus I (baskets) at 100 rpm in 900 mL at 37° C., 0-2 hours, 0.1N HCl (pH 1.2); 2-4 hours, acetate buffer (pH 4.5); 4-12 hours, phosphate buffer (pH 6.8).

In embodiments in which the pharmaceutical composition contains an immediate release portion and an extended release portion, the extended release portion releases the remaining total amount of the active agent in the pharmaceutical composition at a slower rate than the release rate of the immediate release portion. In some embodiments, a second rise in the active agent release rate, measured with an in vitro dissolution test, does not occur after 2 hours, after 2.5 hours, after 3 hours, after 3.5 hours, after 4 hours, after 4.5 hours, after 5 hours, after 5.5 hours, or after 6 hours from the start of the dissolution test. In another embodiment, the second rise does not occur between about 4 to about 16 hours, between about 4 to about 15 hours, between about 4 to about 14 hours, between about 4 to about 14 hours, between about 4 to about 12 hours, between about 4 to about 11 hours, between about 4 to about 10 hours, between about 4 to about 9 hours, between about 4 to about 8 hours, between about 4.5 to about 16 hours, between about 4.5 to about 15 hours, between about 4.5 to about 14 hours, between about 4.5 to about 13 hours, between about 4.5 to about 12 hours, between about 4.5 to about 11 hours, between about 4.5 to about 10 hours, between about 4.5 to about 9 hours, between about 4.5 to about 8 hours, between about 5 to about 16 hours, between about 5 to about 15 hours, between about 5 to about 14 hours, between about 5 to about 13 hours, between about 5 to about 12 hours, between about 5 to about 11 hours, between about 5 to about 10 hours, between about 5 to about 9 hours, between about 5 to about 8 hours, between about 6 to about 16 hours, between about 6 to about 15 hours, between about 6 to about 14 hours, between about 6 to about 13 hours, between about 6 to about 12 hours, between about 6 to about 11 hours, between about 6 to about 10 hours, between about 6 to about 9 hours, or between about 6 to about 8 hours after the start of the dissolution test.

In some embodiments, a second rise in the active agent release rate, measured with an in vitro dissolution test, does occur after 2 hours, after 2.5 hours, after 3 hours, after 3.5 hours, after 4 hours, after 4.5 hours from the start of the dissolution test, but does not occur after 5 hours from the start of the dissolution test. In another embodiment, the second rise does occur between about 3 to about 5 hours, between about 3 to about 4.5 hours, between about 3.5 to about 4.5 hours, or between about 4 to about 4.5 hours after the start of the dissolution test.

In some embodiments, the release rate of the active agent does not substantially decrease during the extended release period where, for example, the decrease in release rate of the extended release portion is less than the decrease in release rate of the immediate release portion (e.g., taking place about 15 min to about 1 hour after the start of an in vitro dissolution test). In some embodiments, the release rate of the active agent decreases during the extended release period less than about 10%/hr, less than about 9%/hr, less than about 8%/hr, less than about 7%/hr, less than about 6%/hr, less than about 5%/hr, less than about 4%/hr, or less than about 3%/hr between about 1 hour to about 16 hours, about 1 hour to about 15 hours, about 1 hour to about 14 hours, about 1 hour to about 13 hours, about 1 hour to about 12 hours, about 1 hour to about 11 hours, about 1 hour to about 10 hours, about 1 hour to about 9 hours, about 1 hour to about 8 hours, about 1.5 hours to about 12 hours, about 1.5 hours to about 11 hours, about 1.5 hours to about 10 hours, about 1.5 hours to about 9 hours, about 1.5 hours to about 8 hours, about 2 hours to about 12 hours, about 2 hours to about 11 hours, about 2 hours to about 10 hours, about 2 hours to about 9 hours, about 2 hours to about 8 hours, about 2.5 hours to about 12 hours, about 2.5 hours to about 11 hours, about 2.5 hours to about 10 hours, about 2.5 hours to about 9 hours, about 2.5 hours to about 8 hours, about 3 hours to about 12 hours, about 3 hours to about 11 hours, about 3 hours to about 10 hours, about 3 hours to about 9 hours, about 3 hours to about 8 hours, about 4 hours to about 12 hours, about 4 hours to about 11 hours, about 4 hours to about 10 hours, about 4 hours to about 9 hours, or about 4 hours to about 8 hours after the start of an in vitro dissolution test.

The invention further provides a pharmaceutical composition comprising: a first release portion and a second release portion, wherein the first release portion and the second release portion each comprise an active agent selected from the group consisting of atomoxetine, a pharmaceutically acceptable salt of atomoxetine, and a combination thereof; wherein the first release portion is an immediate release portion and the second release portion is an extended release portion and the second release portion releases the active agent at a slower rate than the release rate of the first release portion in an in vitro dissolution test; and wherein the composition releases at least about 20% but less than about 45% w/w of the total weight of the active agent within about 1 hour, at least about 40% but less than about 80% of the total weight of the active agent at about 6 hours, and at least 95% of the total weight of the active agent at about 12 hours, as measured by an in vitro dissolution test. In some embodiments, the in vitro dissolution test is performed with USP Apparatus I (baskets) at 100 rpm in 900 mL at 37° C., 0-2 hours, 0.1N HCl (pH 1.2); 2-4 hours, acetate buffer (pH 4.5); 4-12 hours, phosphate buffer (pH 6.8).

In some embodiments, the pharmaceutical compositions disclosed herein allow for in vivo release of an active agent (e.g., atomoxetine hydrochloride) for up to about 6 hours, up to about 8 hours, up to about 10 hours, up to about 12 hours, up to about 14 hours, or up to about 16 hours, after administration to a subject, and can be characterized by a release profile in an in vitro dissolution test comprising (i) a first phase which is an immediate release of the active agent and (ii) a second phase which is an extended release of the active agent. In some embodiments, the second phase release is steady or slower than the first (fast) release. In some embodiments, the second phase comprises a second rise in release rate that takes place about 2 to about 4.5 hours after start of the in vitro dissolution test. In some embodiments, the second phase does not comprise a second rise after 5 hours (e.g., no second rise takes place between 510 hours) from the start of the in vitro dissolution test. In some embodiments, the second phase comprises a third rise in release rate that takes place at least about 6 hours after start of the in vitro dissolution test.

In some embodiments, the first phase is characterized by the release of substantially all, at least about 80%, at least about 85%, at least about 90%, at least about 95%, at least about 96%, at least about 97%, at least about 98%, at least about 99%, or all of the active agent (e.g., atomoxetine hydrochloride) in the immediate release portion within about 2 hours, about 90 minutes, about 1 hour, about 55 minutes, about 50 minutes, about 45 minutes, about 40 minutes, about 35 minutes, about 30 minutes, about 25 minutes, about 20 minutes, or about 15 minutes after the start of an in vitro dissolution test.

In some embodiments, the first phase is characterized by the release of about 15% to about 55%, about 20% to about 55%, about 20% to about 50%, about 20% to about 45%, about 20% to about 40%, about 25% to about 40%, about 30% to about 50%, or about 30% to about 40% (e.g., 15-55%, 20-55%, 20-50%, 20-45%, 20-40%, 25-40%, 30-50%, or 30-40%) of the total weight of the active agent (e.g., atomoxetine hydrochloride) in the composition within about 2 hours, about 90 minutes, about 1 hour, about 55 minutes, about 50 minutes, about 45 minutes, about 40 minutes, about 35 minutes, about 30 minutes, about 25 minutes, about 20 minutes, or about 15 minutes after the start of an in vitro dissolution test. In another embodiment, the first phase is characterized by a release of about 15% to about 55%, about 20% to about 55%, about 20% to about 50%, about 20% to about 45%, about 20% to about 40%, about 25% to about 40%, about 30% to about 50%, or about 30% to about 40% (e.g., 15-55%, 20-55%, 20-50%, 20-45%, 20-40%, 25-40%, 30-50%, or 30-40%) of the total weight of active agent in the composition in less than 1 hour, less than 55 minutes, less than 50 minutes, less than 45 minutes, less than 40 minutes, less than 35 minutes, less than 30 minutes, less than 25 minutes, less than 20 minutes, or less than 15 minutes after the start of an in vitro dissolution test.

In another embodiment, the second phase is characterized by the release of substantially all, at least about 80%, at least about 85%, at least about 90%, at least about 95%, at least about 96%, at least about 97%, at least about 98%, at least about 99%, or all of the active agent (e.g., atomoxetine hydrochloride) in the extended release portion over an extended or prolonged duration of about 8 to about 16 hours, about 10 to about 16 hours, about 12 to about 16 hours, about 14 to about 16 hours, about 8 to about 14 hours, about 8 to about 12 hours, about 8 to about 10 hours, about 10 to about 14 hours, about 10 to about 12 hours, or about 12 to about 14 hours (e.g., about 16 hours, about 15 hours, about 14 hours, about 13 hours, about 12 hours, about 11 hours, about 10 hours, or about 8 hours) after the start of an in vitro dissolution test. In one embodiment, the second phase is characterized by the release of substantially all, at least about 80%, at least about 85%, at least about 90%, at least about 95%, at least about 96%, at least about 97%, at least about 98%, at least about 99%, or all of the active agent in the extended release portion over a duration of between about 8 to about 12 hours, about 9 to about 12 hours, about 10 to about 12 hours, about 11 to about 12 hours, about 8 hours, about 9, hours, about 10 hours, about 11 hours, about 12 hours, about 13 hours, or about 14 hours after the start of an in vitro dissolution test.

In some embodiments, the first phase is relatively fast and the second phase is steady or slower than the first phase with no second rise about 4 hours, about 5 hours, about 6 hours, about 7 hours, about 8 hours, about 9 hours, about 10 hours, about 11 hours, about 12 hours, between about 4 to about 12 hours, between about 4 to about 11 hours, between about 4 to about 10 hours, between about 4 to about 9 hours, between about 4 to about 8 hours, between about 4.5 to about 12 hours, between about 4.5 to about 11 hours, between about 4.5 to about 10 hours, between about 4.5 to about 9 hours, between about 4.5 to about 8 hours, between about 5 to about 12 hours, between about 5 to about 11 hours, between about 5 to about 10 hours, between about 5 to about 9 hours, between about 5 to about 8 hours, between about 6 to about 12 hours, between about 6 to about 11 hours, between about 6 to about 10 hours, between about 6 to about 9 hours, or between about 6 to about 8 hours after the start of an in vitro dissolution test.

In some embodiments, the first phase is relatively fast and the second phase is steady or slower than the first phase with a second rise in release rate that takes place: about 3 hours to about 5 hours, about 3 hours to about 4.5 hours, about 3.5 to about 4.5 hours, about 4 to about 4.5 hours, about 4 hours, about 4.5 hours, prior to about 5 hours, or prior to 5 hours after the start of an in vitro dissolution test.

### Methods of Use

The present disclosure provides methods of using the pharmaceutical compositions disclosed herein. Some aspects of the disclosure are directed to a method of treating or reducing the incidence of orthostatic hypotension in a human subject in need thereof comprising administering to the subject a pharmaceutical composition described herein to the subject. The active agent in the composition can include one or more of: atomoxetine, a pharmaceutically acceptable salt of atomoxetine, or a combination thereof.

In some embodiments, the pharmaceutical compositions disclosed herein increases systolic blood pressure measured one minute after standing. In some embodiments, the pharmaceutical composition maintains blood pressure of the subject within desired levels throughout the day, e.g., greater than 90 mmHg systolic and greater than 60 mmHg diastolic, e.g., about 120/80 mmHg.

In some embodiments, the invention provides a method for treating or reducing the incidence in a subject of one or more conditions selected from the group consisting of: orthostatic hypotension, postural orthostatic tachycardia syndrome (POTS), vasovagal syncope, dysautonomia, retrograde ejaculation or other disorder of semen ejaculation, symptoms of chronic orthostatic hypotension corresponding to autonomic failure associated with Bradbury-Eggleston, Shy-Drager syndromes, diabetes mellitus disease, Parkinson's disease, comprising administering the pharmaceutical composition of the invention to a subject having the condition.

In some embodiments, the subject is 10-50 years old, 10-25 years old, e.g., 13-18 years old, 13-21 years old, or 13-25 years old. In some embodiments, the subject is male. In some embodiments, the subject is female. In some embodiments, the subject is female, aged 13-25 years old and suffers from POTS. In some embodiments, the subject suffers from Parkinson's disease. In some embodiments, the subject suffers from early-onset Parkinson's disease (e.g., is 50 years old or younger). In some embodiments, the subject is older than 50 years.

In some embodiments, the pharmaceutical composition is an oral suspension and the dosage can be titrated to an effective level for the subject, meaning that a subject can start with an initial daily dose of the active agent (e.g., 18 mg/day) and subsequently, the daily dose can be increased or decreased after assessing the subject's response to the initial daily dose. For example, a subject can be started on a low dose (e.g., about 10 mg or about 18 mg atomoxetine or equivalent amount of a pharmaceutically acceptable salt thereof), and then the dose can be increased if the subject does not respond significantly to the low dose. Alternatively, in some subjects it may be necessary to lower the daily dose to minimize adverse effects. In addition, the dose can be titrated to a subject's weight. Dose titration may be particularly important for children, and lower weight adults. In addition, dose titration may be important for poor metabolizers of atomoxetine (discussed above). Multi-particulate dosage forms, such as those containing pellets or granules, and oral suspensions containing multi-particulates are especially useful for dose titration. For example, dose titration can be conveniently achieved by adjusting the volume of oral suspension to be administered to the subject.

In some embodiments, the methods of the present application allow the active agent (e.g., atomoxetine hydrochloride) to be given less frequently than an immediate release dosage and still maintain blood pressure in a clinically acceptable range, e.g., greater than 90 mmHg systolic and greater than 60 mmHg diastolic, e.g., about 120/80 mmHg, throughout the day.

In one embodiment of the method of the invention, the pharmaceutical composition disclosed herein is administered once per day to a human subject in need thereof

### Dosage

The pharmaceutical compositions disclosed herein can be administered once daily to a human subject in need thereof. The dose administered is effective to cause a desired therapeutic and/or prophylactic response in the subject.

In some embodiments, the daily dose of the active ingredient is about 5 mg to about 150 mg, about 5 mg to about 110 mg, about 7.5 mg to about 150 mg, about 7.5 mg to about 120 mg, about 7.5 mg to about 100 mg, about 7.5 mg to about 75 mg, about 7.5 mg to about 50 mg, about 10 mg to about 120 mg, about 10 mg to about 100 mg, about 10 mg to about 80 mg, about 10 mg to about 40 mg, about 10 mg to about 20 mg, about 10 mg to about 18 mg, about 18 mg to about 100 mg, about 18 mg to about 80 mg, about 18 mg to about 40 mg, about 40 mg to about 100 mg, about 40 mg to about 80 mg, about 80 to about 100 mg, about 20 mg to about 75 mg, about 20 mg to about 50 mg, about 25 mg to about 35 mg, or about 40 mg to about 50 mg.

In some embodiments, the daily dose of the active agent is about 5 mg, about 5.5 mg, about 6 mg, about 6.5 mg, about 7 mg, about 7.5 mg, about 8 mg, about 8.5 mg, about 9 mg, about 9.5 mg, about 10 mg, about 12.5 mg, about 15 mg, about 17.5 mg, about 18 mg, about 20 mg, about 20.5 mg, about 22.5 mg, about 25 mg, about 27.5 mg, about 30 mg, about 32.5 mg, about 35 mg, about 37.5 mg, about 38 mg, about 40 mg, about 41 mg, about 42 mg, about 42.5 mg, about 45 mg, about 47.5 mg, about 48 mg, about 50 mg, about 52.5 mg, about 55 mg, about 57.5 mg, about 60 mg, about 62.5 mg, about 65 mg, about 67.5 mg, about 70 mg, about 72.5 mg, about 75 mg, about 77.5 mg, about 80 mg, about 90 mg, about 100 mg, about 110 mg, about 120 mg, about 130 mg, about 140 mg, or about 150 mg.

In some embodiments, the daily dose of the active agent is about 10 mg, about 18 mg, about 40 mg, about 80 mg, or about 100 mg of atomoxetine, or is an amount of atomoxetine HCl that is equivalent to one of these doses of atomoxetine.

### Methods of Making

In some aspects, the application is directed to making a pharmaceutical composition disclosed herein.

The pharmaceutical compositions disclosed herein can be prepared by any number of manufacturing processes known in the art. Non-limiting examples of suitable manufacturing processes include dry granulation, wet granulation, roller compaction, extrusion/spheronization, rotary pelletization, hot melt extrusion, fluid bed granulation, fluid bed coating, compression coating, powder coating, and the like.

In some embodiments, the pharmaceutical composition is prepared for oral delivery. In some embodiments, the pharmaceutical composition is a tablet, a capsule, an orally disintegrating tablet, a chewable tablet, a buccal adhesive tablet, a sublingual tablet, an oral suspension, or a powder, granules, or multi-particulates for oral suspension.

In certain embodiments, the methods of making comprise making a pharmaceutical composition comprising a multilayer tablet (e.g., a bi-layer tablet or a tri-layer tablet) with immediate release and extended release layers, a tablet with an extended release core and an immediate release drug coating over the extended release core, a tablet-in-tablet with an extended release in an immediate release tablet, a compression coated tablet having an extended release core and an immediate release coat, an immediate release core coated with an extended release coating followed by an immediate release drug coating, a capsule filled with multi-particulates (e.g., pellets, particles, granules, beads, spheres, or mini-tablets) having an extended release core and immediate release coating, a blend of fast and extended release multi-particulates compressed into a tablet, a blend of fast and extended release multi-particulates filled into a sachet for reconstitution, or an extended release osmotic controlled tablet with an immediate release drug layer coating.

In some embodiments, the method for making the pharmaceutical composition comprises a drug layering dispersion comprising the active agent layered on to microcrystalline cellulose spheres in fluid bed processor by bottom spray method. A first portion (e.g., at least about 30%, at least about 40%, at least about 50%, at least about 60%, or about 70%) of the drug layered multi-particulates are coated with a sustained release agent (e.g., ethylcellulose and oleic acid) to form a first extended release multi-particulate portion; a second portion (e.g., at least about 15%, at least about 20%, at least about 25%, or about 20%) of the drug layered multi-particulates are coated with a protective layer (e.g., Eudragit® L 30D 55) to form a second extended release multi-particulate portion; and the immediate release (uncoated drug layered multi-particulates), the first extended release multi-particulates, and the second extended release multi-particulates are lubricated (e.g., with Talc and Magnesium stearate) and filled in to capsules.

In some embodiments, the method for making the pharmaceutical composition comprises one or more of dry granulation, wet granulation, roller compaction, extrusion/spheronization, rotary pelletization, hot melt extrusion, fluid bed granulation, fluid bed coating, compression coating, powder coating, and the like.

### Kit

In one embodiment, a kit is provided. In some embodiments, the kit comprise multi-particulates (e.g., pellets, particles, granules, beads, spheres, or mini-tablets) disclosed herein and a liquid vehicle, wherein the multi-particulates can be combined with a liquid vehicle to form a suspension.

In some embodiments, the kit includes a first pharmaceutical composition and a second pharmaceutical composition, wherein the first and second pharmaceutical compositions comprise the same active agent or different active agents.

In one embodiment, the first pharmaceutical composition comprises an immediate release portion and an extended release portion, and the second pharmaceutical composition comprises an extended release portion, wherein both the immediate release portion and the extended release portions include an active agent selected from the group consisting of atomoxetine, a pharmaceutically acceptable salt of atomoxetine, or a combination thereof. In another embodiment, the first pharmaceutical composition comprises an immediate release portion and the second pharmaceutical composition comprises an extended release portion, wherein both the immediate release portion and the extended release portion include an active agent selected from the group consisting of atomoxetine, a pharmaceutically acceptable salt of atomoxetine, or a combination thereof.

In certain embodiments, the first pharmaceutical composition and second composition are administered to a subject simultaneously. In yet another embodiment, the first composition and second composition are administered to a subject sequentially, wherein the second composition is administered after the first composition. In yet another embodiment, the first composition and second composition are administered to a subject sequentially, wherein the first composition is administered after the second composition.

### Combination Therapy

Certain aspects of the application are directed to a method for treating a subject comprising administering a pharmaceutical composition disclosed herein and a further pharmaceutical composition comprising an additional active agent. The additional active agent can be any active drug substance that can be beneficially used with atomoxetine, a pharmaceutically acceptable salt thereof, or combination thereof. Non-limiting examples of additional active agents are hydrocortisone, fludrocortisone, octreotide, and the like. In some embodiments, the pharmaceutical composition can be administered adjunctively with one or more pharmaceutical compositions containing other active agents, e.g., by simultaneous administration of the active agents in the same dosage form, simultaneous administration of the active agents in separate dosage forms, or separate administration of the active agents.

### EXAMPLES

The disclosure is further illustrated by the following examples which are provided merely to be exemplary and do not limit the scope of the invention. Certain modifications and equivalents will be apparent to those skilled in the art and are intended to be included within the scope of the disclosure. The present disclosure provides, but is not limited to, the following formulation examples.

### Example 1

### Atomoxetine HCl Immediate/Fast Release Core Coated with an Extended Release Coating

A tablet containing 18 mg atomoxetine that is an immediate release core coated with an extended release coating followed by an immediate release drug coating can be prepared using the components shown in TABLE 1. Atomoxetine HCl is blended with microcrystalline cellulose and povidone and lubricated with talc and magnesium stearate. This lubricated blend is compressed into an immediate release core tablet. The tablets are coated with an aqueous dispersion containing water, isopropyl alcohol, ethyl cellulose and hypromellose to form an extended release film.

TABLE 1

| | | |
|---|---|---|
| Ingredient No. | Ingredient | mg/unit |

Immediate/Fast Release Core

| | | |
|---|---|---|
| 1 | Atomoxetine | 18.00 |
| 2 | Microcrystalline Cellulose | 123.00 |
| 3 | Povidone | 7.50 |
| 4 | Purified Water | q.s. |
| 5 | Talc | 0.75 |
| 6 | Magnesium Stearate | 0.75 |
| | Total | 150.00 |

Extended Release Coating

| | | |
|---|---|---|
| 7 | Ethyl cellulose | 19.60 |
| 8 | Hypromellose | 4.90 |
| 9 | Purified Water | q.s. |
| 10 | Isopropyl Alcohol | q.s. |
| 11 | Talc | 0.50 |
| 12 | Total | 25.00 |
| | TOTAL | 175.00 |

### Example 2

### Atomoxetine HCl Extended Release Hydrophilic Matrix Tablet

An extended release dissolution/diffusion controlled hydrophilic matrix tablet containing 18 mg of atomoxetine is prepared using components shown in TABLE 2. Atomoxetine, lactose monohydrate and hypromellose are blended and lubricated using talc and magnesium stearate. The lubricated blend is compressed into tablets.

This is an example of a matrix extended release formulation.

TABLE 2

| No. | Ingredients | mg/tablet |
|---|---|---|
| 1 | Atomoxetine | 18.00 |
| 2 | Hypromellose | 144.00 |
| 3 | Lactose monohydrate | 36.00 |
| 4 | Talc | 1.00 |
| 5 | Magnesium stearate | 1.00 |
| | Final tablet weight | 200.00 |

The in vitro dissolution tests for the tablets of Example 2 were performed under the following conditions: USP Apparatus I (baskets) at 100 rpm in 900 mL at 37° C., 0-2 hours, 0.1N HCl (pH 1.2); 2-4 hours, acetate buffer (pH 4.5); 4-12 hours, phosphate buffer (pH 6.8).

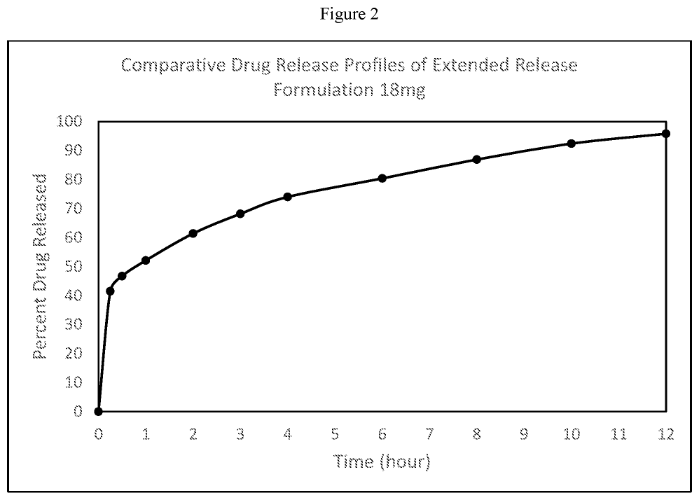

The in vitro dissolution profile for this tablet, plotted as percentage of active agent released over a period of 12 hours, is shown below in Table B, and a graph depicting this profile is shown in FIG. 2. The data in Table B demonstrates that tablets containing 18 mg of atomoxetine provide immediate release of about 52.1% of active agent from the tablet within about 1 hour and an extended release of the remaining active agent over the test period of 12 hours, in vitro.

TABLE B

| Time (hour) | ER Formulation 18 mg |
|---|---|
| 0 | 0 |
| 0.25 | 41.5 |
| 0.5 | 46.7 |
| 1 | 52.1 |
| 2 | 61.4 |
| 3 | 68.2 |
| 4 | 74 |
| 6 | 80.4 |
| 8 | 86.9 |
| 10 | 92.4 |
| 12 | 95.8 |

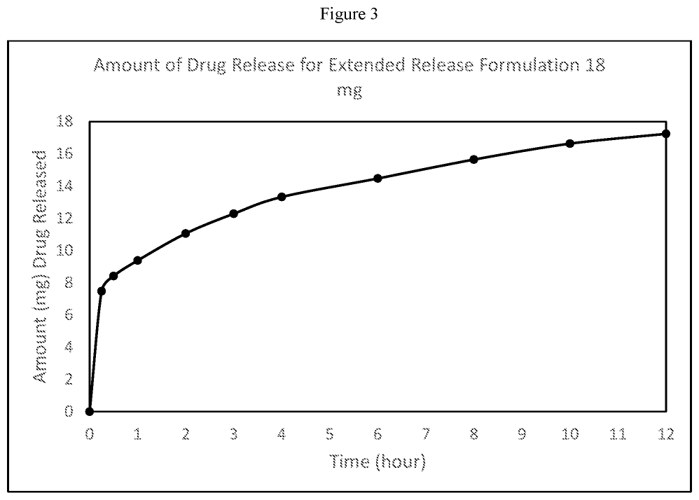

The in vitro dissolution profile for this same tablet formulation, but plotted as amount (mg) of active agent released over a period of 12 hours, is shown below in Table C, and a graph depicting this profile is shown in FIG. 3.

TABLE C

| Time (hour) | ER Formulation 18 mg |
|---|---|
| 0 | 0 |
| 0.25 | 7.47 |
| 0.5 | 8.41 |
| 1 | 9.38 |
| 2 | 11.05 |
| 3 | 12.28 |
| 4 | 13.32 |
| 6 | 14.47 |
| 8 | 15.64 |
| 10 | 16.63 |
| 12 | 17.24 |

The in vitro dissolution data for the tablets tested in Example 2 support the use of the disclosed pharmaceutical formulations in vivo for an effective fast release followed by a steady extended release period.

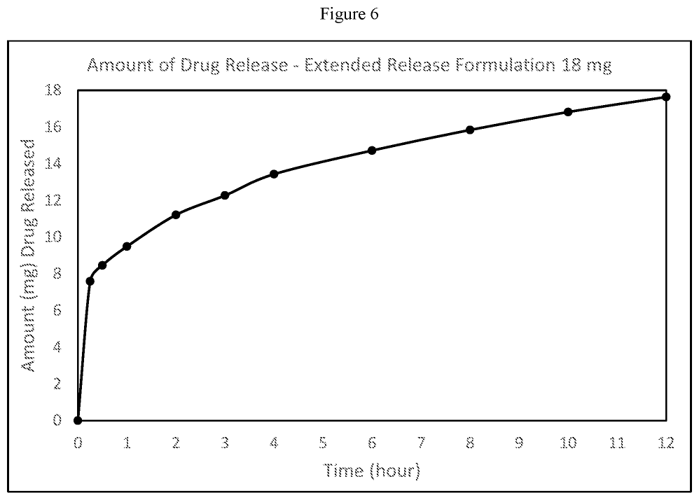

A simulated in vitro dissolution profile for a similar matrix tablet, plotted as percentage of active agent released over a period of 12 hours, is shown below in Table D, and a graph depicting this profile is shown in FIG. 6.

TABLE D

| Time (hour) | ER Formulation 18 mg |
|---|---|
| 0 | 0 |
| 0.25 | 7.59 |
| 0.5 | 8.46 |
| 1 | 9.49 |
| 2 | 11.21 |
| 3 | 12.27 |
| 4 | 13.43 |
| 6 | 14.72 |
| 8 | 15.84 |
| 10 | 16.82 |
| 12 | 17.64 |

### Example 3

### Atomoxetine HCl Extended Release Pellets for Use in Suspension or Capsules

Extended release sachets containing extended release pellets containing 18 mg of atomoxetine can be prepared using components shown in TABLE 3. Atomoxetine HCl is mixed with an aqueous solution of povidone and sprayed on microcrystalline cellulose spheres to form pellets. These pellets are then dried, sized, and coated with a seal coating solution in fluid bed processor by bottom spray method. The seal coated pellets are further coated with an extended release drug coating. These pellets are then blended with mannitol, xanthan gum, mint flavor and lubricated with magnesium stearate. The blend is then filled into sachets. The content of sachet is reconstituted with water at the time of administration to form an extended release suspension.

TABLE 3

| No. | Ingredients | mg/sachet |
|---|---|---|
| | Drug Layering | |
| 1 | Microcrystalline cellulose spheres | 100.00 |
| 2 | Atomoxetine | 18.00 |
| 3 | Povidone | 3.00 |
| 4 | Purified water | q.s. |
| | Total | 121.00 |
| | Seal coating | |
| 5 | Hypromellose | 34.00 |
| 6 | Purified water | q.s. |
| | Total | 34.00 |
| | Sub total | 155.00 |
| | Extended Release Coating | |
| 7 | Ethyl Cellulose | 19.60 |
| 8 | Hypromellose | 4.90 |
| 9 | Purified Water | q.s. |
| 10 | Isopropyl Alcohol | q.s. |
| 11 | Talc | 0.50 |
| | Total | 25.00 |
| | Sub total | 180.00 |
| | Suspending Vehicle Blend | |
| 12 | Mannitol | 302.50 |
| 13 | Xanthan gum | 1.00 |
| 14 | Mint Flavor | 15.00 |
| 15 | Magnesium stearate | 1.50 |
| | Total | 320.00 |
| | TOTAL | 500.00 |

### Example 4

### Atomoxetine HCl Bi-Layer Tablets

Bi-layer tablets having an immediate release layer and an extended release layer were prepared using the components shown in TABLE 4. Atomoxetine HCl was blended with microcrystalline cellulose, croscarmellose sodium and povidone. The blend was then lubricated using colloidal silicon dioxide and magnesium stearate. For the extended release layer, atomoxetine HCl, hypromellose, and microcrystalline cellulose were mixed and granulated using purified water. The granules were dried and lubricated using magnesium stearate. Immediate release and extended release layers were compressed into bi-layer tablets using a bi-layer tablet press.

TABLE 4

| | | 15 mg | 18 mg | 20 mg |
|---|---|---|---|---|
| | | tablet | tablet | tablet |
| Fast/Immediate Release Layer | | | | |
| 1 | Atomoxetine | 5.00 | 6.00 | 7.00 |
| 2 | Microcrystalline | 59.00 | 58.00 | 57.00 |
| | cellulose | | | |
| 3 | Croscarmellose Sodium | 10.00 | 10.00 | 10.00 |
| 4 | Povidone | 5.0 | 5.0 | 5.0 |
| 5 | Colloidal Silicon | 0.50 | 0.50 | 0.50 |
| | Dioxide | | | |
| 6 | Magnesium Stearate | 0.50 | 0.50 | 0.50 |
| | Total | 80.00 | 80.00 | 80.00 |
| Extended Release Layer | | | | |
| 7 | Atomoxetine | 10.00 | 12.00 | 13.00 |
| 8 | Microcrystalline | 53.00 | 51.00 | 50.00 |
| | cellulose | | | |
| 9 | Hydroxy propyl | 96.00 | 96.00 | 96.00 |
| | cellulose (hypromellose) | | | |
| 10 | Magnesium Stearate | 1.00 | 1.00 | 1.00 |
| | Total | 160.00 | 160.00 | 160.00 |
| | TOTAL | 240.00 | 240.00 | 240.00 |

The in vitro dissolution tests for the 18 mg bi-layer tablets of Example 4 were performed under the following conditions: USP Apparatus I (baskets) at 100 rpm in 900 mL at 37° C., 0-2 hours, 0.1N HCl (pH 1.2); 2-4 hours, acetate buffer (pH 4.5); 4-8 hours, phosphate buffer (pH 6.8).

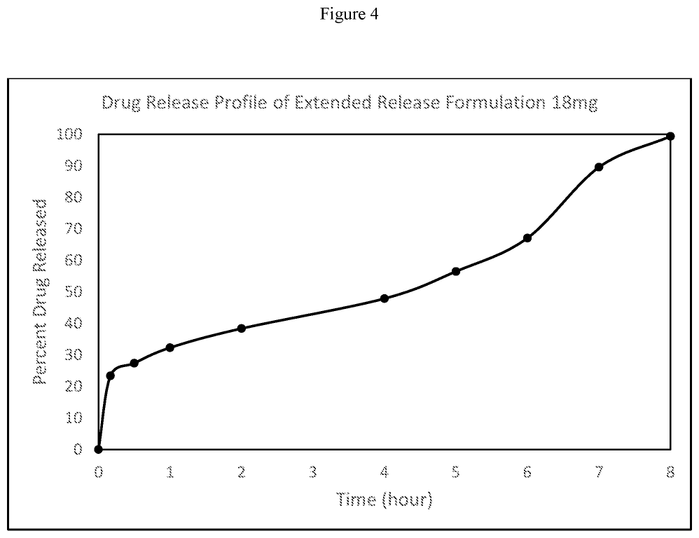

The in vitro dissolution profile for this tablet, plotted as percentage of active agent released for a period of 8 hours, is shown in Table E, below, and a graph depicting this profile is shown in FIG. 4. The data in Table E shows that tablets containing 18 mg of atomoxetine demonstrate immediate release of about 32.3% of active agent from the immediate release layer within about 1 hour and an extended release of the remaining active agent over the test period of 8 hours.

TABLE E

| Time (hour) | ER Formulation 18 mg |
|---|---|
| 0 | 0 |
| 0.167 | 23.4 |
| 0.5 | 27.4 |
| 1 | 32.3 |
| 2 | 38.4 |
| 4 | 47.9 |
| 5 | 56.5 |
| 6 | 67.1 |
| 7 | 89.6 |
| 8 | 99.4 |

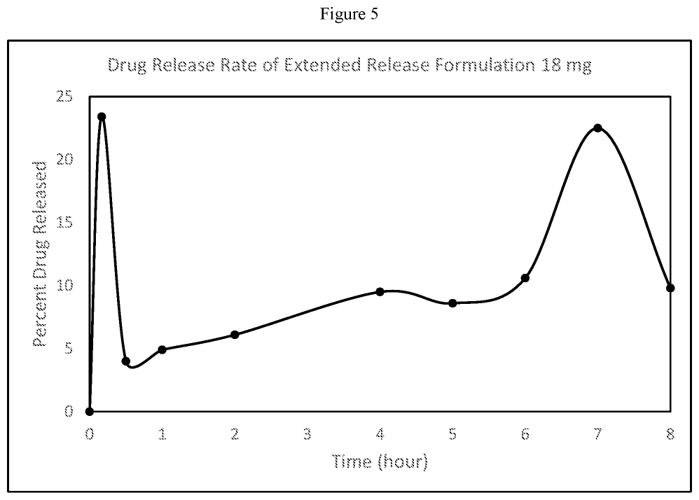

The in vitro dissolution profile for this tablet, plotted as percent active agent released since the previous sample time point, over a period of 8 hours, is shown in Table F, below, and a graph depicting this profile is shown in FIG. 5. The graph in FIG. 5 shows that atomoxetine is released rapidly, followed by a sustained release of the drug, followed by a rapid release of the drug.

TABLE F

| Time (hour) | ER Formulation 18 mg |
|---|---|
| 0 | 0 |
| 0.167 | 23.4 |
| 0.5 | 4 |
| 1 | 4.9 |
| 2 | 6.1 |
| 4 | 9.5 |
| 5 | 8.6 |
| 6 | 10.6 |
| 7 | 22.5 |
| 8 | 9.8 |

The in vitro dissolution data for the bi-layer tablets tested in Example 4 support the use of the disclosed pharmaceutical formulations in vivo for an effective immediate release followed by an extended release period.

### Example 5

### Atomoxetine HCl Extended Release Ion-Exchange Complex Sachets

Sachets of an extended release Atomoxetine-ion exchange resin complex can be prepared using the components shown in TABLE 5. The complex is formed by mixing atomoxetine with ion exchange resin in water and then dried. The complex can then blended with mannitol, xanthan gum, mint flavor and lubricated with magnesium stearate. The blend is then filled into sachets. The content of sachet is reconstituted with water at the time of administration to form an extended release suspension.

TABLE 5

| | | |
|---|---|---|
| Ingredient No. | Ingredient | mg/sachet |

Extended Release Complex

| | | |
|---|---|---|
| 1 | Atomoxetine | 18.00 |
| 2 | Polyacrilin Potassium | 36.00 |
| 3 | Purified water | q.s. |
| 4 | Talc | 1.00 |
| | Total | 55.00 |

Suspending Vehicle Blend

| | | |
|---|---|---|
| 5 | Mannitol | 277.50 |
| 6 | Xanthan Gum | 1.00 |
| 7 | Orange Flavor | 15.00 |
| | Magnesium Stearate | 1.50 |
| | Total | 295.00 |
| | TOTAL | 350.00 |

### Example 6

### Atomoxetine HCl Floating Drug Delivery System

An extended release floating tablet containing 18 mg of atomoxetine is prepared using components shown in TABLE 6. Atomoxetine HCl, hydroxyethyl cellulose, lactose monohydrate and Hypromellose, ethyl cellulose and sodium bicarbonate are blended in a high shear granulator. The blend is granulated by a wet granulation method using isopropyl alcohol. The granules are dried, milled and lubricated using talc and magnesium stearate. The lubricated blend is compressed into tablets.

TABLE 6

| No. | Ingredients | mg/tablet |
|---|---|---|
| 1 | Atomoxetine | 18.00 |
| 2 | Hydroxyethyl Cellulose | 80.00 |
| 3 | Lactose Monohydrate | 48.00 |
| 4 | Ethyl Cellulose | 10.00 |
| 5 | Sodium Bicarbonate | 40.00 |
| 6 | Isopropyl Alcohol | q.s. |
| 7 | Talc | 2.00 |
| 8 | Magnesium stearate | 2.00 |
| | Total | 200.00 |

### Example 7

### Atomoxetine HCl Immediate Release Core Coated with an Extended Release Coating and Immediate Release Drug Coating

An immediate release core coated with an extended release coating followed by an immediate release drug coating can be prepared using the components shown in TABLE 7. Atomoxetine HCl is blended with microcrystalline cellulose and lubricated with colloidal silicone dioxide and magnesium stearate. This lubricated blend is compressed into an immediate release core tablet. The tablets are coated with ethyl cellulose aqueous dispersion and hypromellose to form an extended release film. The coated extended release tablets are further coated with an aqueous solution of Atomoxetine HCl and hypromellose to form an outer immediate release drug coat on the tablets.

TABLE 7

| | | |
|---|---|---|
| Ingredient No. | Ingredient | mg/unit |

Immediate Release Core

| | | |
|---|---|---|
| 1 | Atomoxetine | 6.00 |
| 2 | Microcrystalline Cellulose | 91.50 |
| 3 | Colloidal Silicon Dioxide | 1.50 |
| 4 | Magnesium Stearate | 1.00 |
| | Total | 100.00 |

Extended Release Coating

| | | |
|---|---|---|
| 5 | Ethyl cellulose aqueous dispersion | 9.60 |
| 6 | Hypromellose | 6.40 |
| 7 | Purified Water | QS |
| 8 | Total | 16.00 |

Immediate Release Drug Coating

| | | |
|---|---|---|
| 9 | Atomoxetine | 12.00 |
| 10 | Hypromellose | 12.00 |
| 11 | Purified water | QS |
| 12 | Total | 24.00 |
| | TOTAL | 140.00 |

### Example 8

### Atomoxetine HCl Capsule Filled with Pellets Having an Extended Release Core and Immediate Release Coating

A capsule filled with pellets having an extended release core and immediate release coating can be prepared using the components shown in TABLE 8. Atomoxetine HCl, microcrystalline cellulose and hypromellose are blended and granulated using aqueous solution of povidone in a high shear granulator. The wet mass is extruded and spheronized using extrusion and spheronizer to form wet spherical pellets. These extended release matrix pellets are dried in a fluid bed dryer and further coated with an aqueous solution of atomoxetine HCl and hypromellose to form an outer immediate release drug layer. These pellets are filled into a size 1 capsule using capsule filling machine.

TABLE 8

| | | |
|---|---|---|
| Ingredient No. | Ingredient | mg/capsule |

Extended Release Core

| | | |
|---|---|---|
| 1 | Atomoxetine | 16.00 |
| 2 | Microcrystalline Cellulose | 121.00 |
| 3 | Hypromellose | 203.00 |
| 4 | Povidone | 10.00 |
| 5 | Purified water | QS |
| | Total | 350.00 |

Immediate Release Drug Coating

| | | |
|---|---|---|
| 6 | Atomoxetine | 4.00 |
| 7 | Hypromellose | 10.00 |
| 8 | Purified water | QS |
| 9 | Total | 14.00 |
| | TOTAL | 364.00 |
| 10 | Size 1 capsule | 96.00 |
| | TOTAL | 460.00 |

### Example 9

### Atomoxetine HCl Bi-Layer Extended Release Tablet Having an Immediate and an Extended Release Layer

A bi-layer extended release tablet with fast and extended release layers can be prepared using components shown in TABLE 9. For the immediate release layer, atomoxetine HCl, microcrystalline cellulose, sodium starch glycolate and iron oxide yellow are blended and lubricated using talc and magnesium stearate. For the extended release layer, atomoxetine HCl, microcrystalline cellulose and hypromellose are blended and lubricated using colloidal silicon dioxide and magnesium stearate. These lubricated layers are then compressed into bi-layer tablets using a bi-layer tablet press.

TABLE 9

| No. | Ingredients | mg/tablet |
|---|---|---|
| | Immediate Release layer | |
| 1 | Atomoxetine | 5.40 |
| 2 | Microcrystalline cellulose | 142.60 |
| 3 | Sodium starch glycolate | 9.60 |
| 4 | Iron oxide yellow | 0.80 |
| 5 | Talc | 0.80 |
| 6 | Magnesium stearate | 0.80 |
| | Total | 160.00 |
| | Extended release layer | |
| 7 | Atomoxetine | 12.60 |
| 8 | Microcrystalline cellulose | 178.20 |
| 9 | Hypromellose | 126.00 |
| 10 | Colloidal silicon dioxide | 1.60 |
| 11 | Magnesium stearate | 1.60 |
| | Total | 320.00 |
| | Final tablet weight | 480.00 |

### Example 10

### Atomoxetine HCl Tri-Layer Extended Release Tablet

A tri-layer extended release tablet with one fast layer and two extended release layers can be prepared using components shown in TABLE 10. For the immediate release layer, atomoxetine HCl, microcrystalline cellulose, crospovidone and iron oxide yellow are blended and lubricated using talc and magnesium stearate. For the first extended release layer, Atomoxetine HCl, microcrystalline cellulose, hypromellose and iron oxide red are blended and lubricated using colloidal silicon dioxide and magnesium stearate. For the second extended release layer, Atomoxetine HCl, microcrystalline cellulose and hypromellose are blended and lubricated using colloidal silicon dioxide and magnesium stearate. These lubricated layers are then compressed into tri-layer tablets using a multi-layer tablet press.

TABLE 10

| No. | Ingredients | mg/tablet |
|---|---|---|
| | Immediate Release layer | |
| 1 | Atomoxetine | 5.00 |
| 2 | Microcrystalline cellulose | 143.00 |
| 3 | Crospovidone | 9.60 |
| 4 | Iron oxide yellow | 0.80 |
| 5 | Talc | 0.80 |
| 6 | Magnesium stearate | 0.80 |
| | Total | 160.00 |
| | 1^{st} Extended release layer | |
| 7 | Atomoxetine | 10.00 |
| 8 | Microcrystalline cellulose | 99.60 |
| 9 | Hypromellose | 48.00 |
| 10 | Iron oxide red | 0.80 |
| 11 | Colloidal silicon dioxide | 0.80 |
| 12 | Magnesium stearate | 0.80 |
| | Total | 160.00 |
| | 2^{nd} Extended release layer | |
| 13 | Atomoxetine | 5.00 |
| 14 | Microcrystalline cellulose | 133.40 |
| 15 | Eudragit ® L 30D 55 (30% w/w aqueous dispersion) | 20.00 |
| 16 | Colloidal silicon dioxide | 0.80 |
| 17 | Magnesium stearate | 0.80 |
| | Total | 160.00 |
| | Final tablet weight | 480.00 |

### Example 11

### Atomoxetine HCl Extended Release Hydrophilic Matrix with Immediate Release Drug Coat

A tablet containing 18 mg of atomoxetine with an extended release hydrophilic matrix core and an immediate release drug coat over the extended release core can be prepared using components shown in TABLE 11. Atomoxetine HCl, microcrystalline cellulose and hydroxypropyl cellulose are blended in a high shear granulator. The blend is granulated by a wet granulation method using purified water. The granules are dried, milled and lubricated using colloidal silicon dioxide and magnesium stearate. The lubricated blend is compressed into extended release core tablets. These extended release core tablets are coated with an immediate release drug coat solution containing atomoxetine HCl, hypromellose, polyethylene glycol and purified water in a perforated coating pan.

TABLE 11

| No. | Ingredients | mg/tablet |
|---|---|---|
| | Extended release core | |
| 1 | Atomoxetine | 12.00 |
| 2 | Microcrystalline cellulose | 120.50 |
| 3 | Hydroxypropyl cellulose | 105.00 |
| 4 | Purified water | QS |
| 5 | Colloidal silicon dioxide | 1.25 |
| 6 | Magnesium stearate | 1.25 |
| | Total | 240.00 |
| | Immediate release drug coat | |
| 7 | Atomoxetine | 6.00 |
| 8 | Hypromellose | 12.80 |
| 9 | Polyethylene glycol | 1.20 |
| 10 | Purified water | QS |
| | Total | 20.00 |
| | Final tablet weight | 260.00 |

### Example 12

### Atomoxetine HCl Extended Release Hydrophilic Matrix Coated with a Water-Permeable Functional Coat Followed by an Immediate Release Drug Coat

A tablet having an extended release hydrophilic matrix core, a water-permeable functional coating, and an immediate release drug coating is prepared using components shown in TABLE 12. Atomoxetine HCl, lactose monohydrate and hypromellose are blended and lubricated using talc and magnesium stearate. The lubricated blend is compressed into extended release core tablets. These tablets are coated with a functional coating containing an aqueous dispersion of Surelease® and Opadry® Clear. The tablets are further coated with an immediate release drug coating containing atomoxetine HCl, croscarmellose sodium, hydroxypropyl cellulose, polyethylene glycol, and purified water.

TABLE 12

| No. | Ingredients | mg/tablet |
|---|---|---|
| | Extended release core | |
| 1 | Atomoxetine | 15.00 |
| 2 | Lactose monohydrate | 37.00 |
| 3 | Hypromellose | 146.00 |
| 4 | Talc | 1.00 |
| 5 | Magnesium stearate | 1.00 |
| | Total | 200.00 |
| | Functional coat | |
| 6 | Surelease ® (30% w/w aqueous dispersion) | 9.00 |
| 7 | Opadry ® Clear | 6.00 |
| 8 | Purified water | QS |
| | Total | 15.00 |
| | Sub total | 215.00 |
| | Immediate release drug coat | |
| 9 | Atomoxetine | 5.00 |
| 10 | Croscarmellose Sodium | 4.00 |
| 11 | Hydroxypropyl cellulose | 14.60 |
| 12 | Polyethylene glycol | 1.40 |
| 13 | Purified water | QS |
| | Total | 25.00 |
| | Final tablet weight | 240.00 |

### Example 13

### Atomoxetine HCl Tablet-In-Tablet with an Extended Release Core Covered with an Immediate Release Drug Coat

A tablet-in-tablet with an extended release tablet in an immediate release tablet can be prepared using components shown in TABLE 13. For the extended release core tablet, atomoxetine HCl and lactose monohydrate are blended and granulated using Surelease® by top spray granulation in a fluid bed processor. The granules are dried, milled and lubricated using talc and magnesium stearate. For the immediate release outer tablet, a blend of atomoxetine HCl, crospovidone and iron oxide red is geometrically mixed with microcrystalline cellulose. The blend is then lubricated with talc and magnesium stearate. Using a specially designed tablet-in-tablet compression machine, the extended release core tablet is compressed followed by compression of the outer immediate release tablet around the extended release core tablet.

TABLE 13

| No. | Ingredients | mg/tablet |
|---|---|---|
| | Extended release core | |
| 1 | Atomoxetine | 12.00 |
| 2 | Lactose monohydrate | 57.00 |
| 3 | Surelease ® (30% w/w aqueous dispersion) | 30.00 |
| 4 | Talc | 0.50 |
| 5 | Magnesium stearate | 0.50 |
| | Total | 100.00 |
| | Immediate release outer coat | |
| 6 | Atomoxetine | 3.00 |
| 7 | Microcrystalline cellulose | 182.80 |
| 8 | Crospovidone | 12.00 |
| 9 | Iron oxide red | 0.20 |
| 10 | Talc | 1.00 |
| 11 | Magnesium stearate | 1.00 |
| | Total | 200.00 |
| | Final tablet weight | 300.00 |

### Example 14

### Atomoxetine HCl Extended Release Hydrophobic Core Coated with an Immediate Release Drug Coat

A compression coated tablet having an extended release hydrophobic core and an immediate release outer coating can be prepared using components shown in TABLE 14. For the extended release core tablet, atomoxetine HCl, microcrystalline cellulose and hydrogenated castor oil are blended in steam jacketed high shear mixer granulator. The blend is then granulated by melting hydrogenated castor oil with the help of steam. The granules are cooled, milled, and lubricated using sodium stearyl fumarate. For the immediate release outer coat tablet, a blend of atomoxetine HCl, sodium starch glycolate and iron oxide red is geometrically mixed with microcrystalline cellulose. The blend is then lubricated with talc and magnesium stearate. Using a specially designed compression machine, the extended release core tablet is compressed followed by compression of the outer immediate release coat tablet.

TABLE 14

| No. | Ingredients | mg/tablet |
|---|---|---|
| | Extended release core | |
| 1 | Atomoxetine | 16.00 |
| 2 | Microcrystalline cellulose | 8.00 |
| 3 | Hydrogenated castor oil | 94.50 |
| 4 | Sodium stearyl fumarate | 1.50 |
| | Total | 120.00 |
| | Immediate release outer coat | |
| 5 | Atomoxetine | 4.00 |
| 6 | Microcrystalline cellulose | 108.30 |
| 7 | Sodium starch glycolate | 6.00 |
| 8 | Iron oxide red | 0.20 |
| 9 | Talc | 0.75 |
| 10 | Magnesium stearate | 0.75 |
| | Total | 120.00 |
| | Final tablet weight | 240.00 |

### Example 15

Atomoxetine HCl Extended Release Capsule Containing Fast and Extended Release Pellets, Prepared by Drug Layering and Functional Coating Method

An extended release capsule containing immediate release pellets and functional coated extended release pellets can be prepared using components shown in TABLE 15. A drug layering dispersion containing atomoxetine HCl, hypromellose, and talc in purified water is layered on to microcrystalline cellulose spheres in fluid bed processor by bottom spray method to prepare immediate release pellets. A portion of these drug-layered pellets is coated with an ethanolic solution of ethylcellulose and oleic acid to form extended release pellets. Then the immediate release and extended release pellets are lubricated with talc and magnesium stearate and filled into capsules.

TABLE 15

| No. | Ingredient | mg/capsule |
|---|---|---|
| | Immediate release pellets | |
| 1 | Microcrystalline cellulose spheres | 74.00 |
| 2 | Atomoxetine | 15.00 |
| 3 | Hypromellose | 10.00 |
| 4 | Talc | 1.00 |
| 5 | Purified water | QS |
| | Total | 100.00 |
| | Extended release pellets | |
| 6 | Immediate release pellets | 60.00 |
| 7 | Ethylcellulose | 10.00 |
| 8 | Oleic acid | 2.00 |
| 9 | Ethanol | QS |
| | Total | 72.00 |
| | Capsule filling | |
| 10 | Immediate release pellets | 40.00 |
| 11 | Extended release pellets | 72.00 |
| 12 | Talc | 1.50 |
| 13 | Magnesium stearate | 0.50 |
| 14 | Size 2 capsule | 60.00 |
| | Filled capsule weight | 174.00 |

### Example 16

### Atomoxetine Extended Release Capsule Containing Fast and Extended Release Pellets

An extended release capsule containing immediate release pellets and functional coated extended release pellets can be prepared using components shown in TABLE 16. A drug layering dispersion containing atomoxetine HCl, hypromellose and talc in purified water is layered on to microcrystalline cellulose spheres in fluid bed processor by bottom spray method to prepare immediate release pellets. One portion (50%) of these drug-layered pellets is coated with an ethanolic solution of ethylcellulose and oleic acid to form a first population of extended release pellets. A second portion (20%) of the drug-layered pellets is coated with an aqueous dispersion of Eudragit® L 30D 55 to form a second population of extended release pellets. Then the immediate release and extended release pellets are lubricated with talc and magnesium stearate and filled into capsules.

TABLE 16

| No. | Ingredient | mg/capsule |
|---|---|---|
| | Immediate release pellets | |
| 1 | Microcrystalline cellulose spheres | 69.00 |
| 2 | Atomoxetine | 20.00 |
| 3 | Hypromellose | 10.00 |
| 4 | Talc | 1.00 |
| 5 | Purified water | QS |
| | Total | 100.00 |
| | 1^{st} Extended release pellets | |
| 6 | Immediate release pellets | 50.00 |
| 7 | Ethylcellulose | 9.00 |
| 8 | Oleic acid | 1.00 |
| 9 | Ethanol | QS |
| | Total | 60.00 |
| | 2^{nd} Extended release pellets | |
| 10 | Immediate release pellets | 20.00 |
| 11 | Eudragit ® L 30D 55 (30% w/w aqueous | 20.00 |
| | dispersion) | |
| 12 | Purified water | QS |
| | Total | 40.00 |
| | Capsule filling | |
| 13 | Immediate release pellets | 30.00 |
| 14 | 1^{st} Extended release pellets | 60.00 |
| 15 | 2^{nd} Extended release pellets | 40.00 |
| 16 | Talc | 1.50 |
| 17 | Magnesium stearate | 0.50 |
| 18 | Size 1 capsule | 76.00 |
| | Filled capsule weight | 208.00 |

### Example 17

### Atomoxetine HCl Orally Disintegrating Extended Release Tablet Containing Immediate and Extended Release Pellets

An orally disintegrating extended release tablet containing immediate release and functional coated extended release pellets can be prepared using components shown in TABLE 17. A drug layering dispersion containing atomoxetine HCl, hypromellose and talc in purified water is layered on to microcrystalline cellulose spheres in fluid bed processor by bottom spray method to prepare drug-layered pellets. These pellets are then coated with an ethanolic solution of ethylcellulose and oleic acid to form extended release pellets. These extended release pellets are further coated with an immediate release drug coating using aqueous dispersion containing atomoxetine HCl, hypromellose and talc. The pellets are then blended with microcrystalline cellulose, mannitol, croscarmellose sodium, aspartame and peppermint flavor and lubricated Magnesium stearate. The lubricated blend is then compressed into orally disintegrating tablets.

TABLE 17

| No. | Ingredients | mg/tablet |
|---|---|---|
| | Drug layered pellets | |
| 1 | Microcrystalline cellulose spheres | 44.50 |
| 2 | Atomoxetine | 9.00 |
| 3 | Hypromellose | 6.00 |
| 4 | Talc | 0.50 |
| 5 | Purified water | QS |
| | Total | 60.00 |
| | Functional coating | |
| 6 | Ethylcellulose | 10.00 |
| 7 | Oleic acid | 2.00 |
| 8 | Ethanol | QS |
| | Total | 12.00 |
| | Sub total | 72.00 |
| | Immediate release drug coat | |
| 9 | Functional coated extended release pellets | 72.00 |
| 10 | Atomoxetine | 6.00 |
| 11 | Hypromellose | 4.00 |
| 12 | Talc | 1.00 |
| 13 | Purified water | QS |
| | Total | 83.00 |
| | Tableting | |
| 14 | Immediate release drug coated extended release pellets | 83.00 |
| 15 | Microcrystalline cellulose | 161.00 |
| 16 | Mannitol | 108.00 |
| 17 | Croscarmellose sodium | 24.00 |
| 18 | Aspartame | 12.00 |
| 19 | Peppermint flavor | 8.00 |
| 20 | Magnesium stearate | 4.00 |
| | Final tablet weight | 400.00 |

### Example 18

### Atomoxetine HCl Extended Release Sachet for Reconstitution Containing Extended Release Pellets Coated with an Immediate Release Drug Coat

Extended release sachets containing extended release pellets coated with an immediate release drug coat can be prepared using components shown in TABLE 18. Atomoxetine HCl, microcrystalline cellulose and hypromellose are mixed and granulated with a non-aqueous solution of povidone in a high shear granulator. The wet mass is extruded and the extrudes are spheronized to form spherical pellets. These extended release pellets are then dried, sized, and coated with a seal coating solution in fluid bed processor by bottom spray method. The seal coated pellets are further coated with an immediate release drug coating. These pellets are then blended with xanthan gum, mannitol, orange flavor and lubricated with magnesium stearate. The blend is then filled into sachets. The content of sachet is reconstituted with water at the time of administration to form an extended release suspension.

TABLE 18

| No. | Ingredients | mg/sachet |
|---|---|---|
| | Extended release pellets | |
| 1 | Atomoxetine | 12.00 |
| 2 | Microcrystalline cellulose | 88.00 |
| 3 | Hypromellose | 160.00 |
| 4 | Povidone | 20.00 |
| 5 | Isopropyl alcohol | QS |
| | Total | 280.00 |
| | Seal coating | |
| 6 | Opadry ® Clear | 28.00 |
| 7 | Purified water | QS |
| | Total | 28.00 |
| | Sub total | 308.00 |
| | Immediate release drug coat | |
| 8 | Atomoxetine | 6.00 |
| 9 | Hypromellose | 15.00 |
| 10 | Talc | 1.00 |
| 11 | Purified water | QS |
| | Total | 22.00 |
| | Sub total | 330.00 |
| | Sachet filling | |
| 14 | Extended release pellets | 360.00 |
| 15 | Xanthan gum | 18.00 |
| 16 | Mannitol | 207.00 |
| 17 | Orange flavor | 12.00 |
| 18 | Magnesium stearate | 3.00 |
| | Sachet filled weight | 600.00 |

### Example 19

### Atomoxetine HCl Extended Release Tablets Based on Osmotic Drug Delivery Coated with an Immediate Release Drug Coat

Extended release tablets based on osmotic drug delivery can be prepared using components shown in TABLE 19. For the extended release drug layer, Atomoxetine HCl, microcrystalline cellulose, polyethylene oxide are granulated in a fluid bed processor using purified water by top spray granulation. The granules are dried, milled, and lubricated using magnesium stearate. The push layer is also prepared by top spray granulation similar to the extended release drug layer. Both the layers are then compressed into bi-layer tablets using a multi-layer tablet compression machine. These tablets are then coated with a functional coating solution of cellulose acetate and polyethylene glycol in acetone and purified water mixture. These tablets are then passed through a laser drilling machine to drill an orifice in the drug layer side. The tablets are then coated with an aqueous solution of atomoxetine HCl and hypromellose to form an immediate release drug coat.

TABLE 19

| No. | Ingredients | mg/tablet |
|---|---|---|
| | Drug layer | |
| 1 | Atomoxetine | 12.00 |
| 2 | Microcrystalline cellulose | 94.00 |
| 3 | Polyethylene oxide | 133.00 |
| 4 | Purified water | QS |
| 5 | Magnesium stearate | 1.00 |
| | Total | 240.00 |
| | Push layer | |
| 6 | Polyethylene oxide | 204.00 |
| 7 | Sodium chloride | 80.00 |
| 8 | Povidone | 12.00 |
| 9 | Iron oxide red | 3.00 |
| 10 | Purified water | QS |
| 11 | Magnesium stearate | 1.00 |
| | Total | 300.00 |
| | Sub total | 540.00 |
| | Functional coating | |
| 12 | Cellulose acetate | 73.00 |
| 13 | Polyethylene glycol | 9.00 |
| 14 | Acetone | QS |
| 15 | Purified water | QS |
| | Total | 82.00 |
| | Sub total | 622.00 |
| | Immediate release drug coat | |
| 16 | Atomoxetine | 8.00 |
| 17 | Hypromellose | 20.00 |
| 18 | Purified water | QS |
| | Total | 28.00 |
| | Final tablet weight | 650.00 |

What is claimed is:

1. A pharmaceutical composition comprising a sustained release agent and about 5 mg to about 110 mg of an active agent selected from the group consisting of atomoxetine, a pharmaceutically acceptable salt of atomoxetine, and a combination thereof, wherein the composition releases: at least about 30% of the total weight of the active agent by about 2 hours, at least about 30% but less than about 90% of the total weight of the active agent by about 4 hours, at least about 50% but less than about 95% of the total weight of the active agent by about 6 hours, and at least about 70% of the total weight of the active agent by about 8 hours, wherein the release profile is measured by an in vitro dissolution test.

2. The pharmaceutical composition of claim 1, wherein the composition releases: at least about 40% of the total weight of the active agent by about 1 hour, at least about 40% but less than about 75% of the total weight of the active agent by about 2 hours, at least about 50% but less than about 80% of the total weight of the active agent by about 4 hours, and at least about 80% of the total weight of the active agent by about 8 hours, as measured by an in vitro dissolution test.

3. A pharmaceutical composition comprising a sustained release agent and about 5 mg to about 110 mg of an active agent selected from the group consisting of atomoxetine, a pharmaceutically acceptable salt of atomoxetine, and a combination thereof, wherein the composition releases: at least about 20% of the total weight of the active agent by about 2 hours, at least about 25% but less than about 80% of the total weight of the active agent by about 4 hours, at least about 40% but less than about 90% of the total weight of the active agent by about 6 hours, and at least 90% of the total weight of the active agent by about 12 hours, as measured by an in vitro dissolution test.

4. The pharmaceutical composition of claim 3, wherein the composition releases: at least about 20% of the total weight of the active agent by about 1 hour, at least about 25% but less than about 60% of the total weight of the active agent by about 2 hours, at least about 30% but less than about 75% of the total weight of the active agent by about 4 hours, and at least about 80% of the total weight of the active agent by about 8 hours, as measured by an in vitro dissolution test.

5. The pharmaceutical composition of any of the preceding claims, wherein the active agent is atomoxetine HCl.

6. The pharmaceutical composition of claim 1, 2, 3 or 4, wherein the total amount of active agent in the composition is selected from the group consisting of about 10 mg, about 18 mg, about 40 mg, about 80 mg and about 100 mg.

7. The pharmaceutical composition of claim 1, 2, 3 or 4, wherein the active agent is atomoxetine HCl, and the total amount of active agent in the composition is equivalent to an amount of atomoxetine selected from the group consisting of about 10 mg, about 18 mg, about 40 mg, about 80 mg and about 100 mg.

8. The pharmaceutical composition of claim 1, 2, 3 or 4, wherein the active agent is present in an amount of about 4% to about 30% of the total weight of the composition.

9. The pharmaceutical composition of claim 8, wherein the ratio of the amount of active agent to the amount of sustained release agent in the composition is about 1:1 to about 1:30 (w/w).

10. The pharmaceutical composition of claim 1, 2, 3 or 4, wherein the composition comprises an ion-exchange complex.

11. The pharmaceutical composition of claim 1, 2, 3 or 4, wherein the composition is a matrix extended release formulation.

12. The pharmaceutical composition of claim 1, 2, 3 or 4, wherein the composition is a bi-layer tablet.

13. The pharmaceutical composition of claim 1, 2, 3 or 4, wherein the composition is a floating tablet.

14. The pharmaceutical composition of claim 1, 2, 3 or 4, wherein the composition is a suspension.

15. The pharmaceutical composition of claim 1, 2, 3 or 4, wherein the composition comprises a first release portion comprising about 20% to about 40% of the weight of the active agent in the composition; and a second release portion comprising about 40% to about 80% of the weight of the active agent in the composition, wherein the second release portion comprises a sustained release agent.

16. A method for treating or reducing the incidence of orthostatic hypotension (OH) in a subject in need thereof comprising administering the pharmaceutical composition of claim 1, 2, 3 or 4 to the subject.

17. A method for treating or reducing the incidence of postural orthostatic tachycardia syndrome (POTS) in a subject in need thereof comprising administering the pharmaceutical composition of claim 1, 2, 3 or 4 to the subject.

18. A method comprising administering the pharmaceutical composition of claim 1, 2, 3 or 4 to a subject to treat or reduce the incidence of a condition selected from the group consisting of: orthostatic hypotension, postural orthostatic tachycardia syndrome (POTS), vasovagal syncope, dysautonomia, retrograde ejaculation or other disorder of semen ejaculation, symptoms of chronic orthostatic hypotension corresponding to autonomic failure associated with Bradbury-Eggleston, Shy-Drager syndromes, diabetes mellitus disease, and Parkinson's disease; in the subject.

19. A method for treating or reducing the incidence of vasovagal syncope in a subject in need thereof comprising administering the pharmaceutical composition of claim 1, 2, 3 or 4 to the subject.

20. The method of claim 16, wherein the unit dose administered to the subject is titrated after assessing the effect of an initial daily dose on the subject.

21. The method of claim 18, wherein the unit dose administered to the subject is titrated after assessing the effect of an initial daily dose on the subject.

22. The method of claim 21, wherein the pharmaceutical composition administered is a suspension.

23. The method of claim 16, 17, 18 or 19, wherein the active agent in the composition is atomoxetine HCl, and the total amount of active agent in the composition is equivalent to an amount of atomoxetine selected from the group consisting of about 10 mg, about 18 mg, about 40 mg, about 80 mg and about 100 mg.

24. The pharmaceutical composition of claim 1, 2, 3 or 4, wherein the in vitro release rate of the active agent from the composition decreases within about 1 hour of the start of the in vitro dissolution test, increases between about 1 to about 4 hours and increases between about 4 and about 8 hours after start of the in vitro dissolution test.

25. The pharmaceutical composition of claim 15, wherein the composition comprises a third release portion that comprises a sustained release agent and an active agent selected from the group consisting of atomoxetine, a pharmaceutically acceptable salt of atomoxetine, and a combination thereof.

26. The pharmaceutical composition of claim 1, 2, 3, or 4, wherein the in vitro release rate of the active agent within the last 4 hours of release is slower than the in vitro release rate of the active agent released during the first 4 hours of release.

27. The pharmaceutical composition of claim 1, 2, 3 or 4, wherein the sustained release agent is selected from the group consisting of hypromellose, hydroxypropyl cellulose, hydroxyethyl cellulose, polyethylene oxide, polyvinylpyrrolidone, polyvinyl alcohol, xanthan gum, guar gum, chitosan, a chitosan derivative, carbomer, carrageenan, carboxymethyl cellulose, sodium alginate, a polyglycolized glyceride, polyethyleneglycol, polyacrilin potassium polyvinyl acetate dispersion, ethyl cellulose, cellulose acetate, cellulose acetate butyrate, cellulose acetate phthalate, cellulose triacetate, beeswax, carnauba wax, paraffin wax, microcrystalline wax, ozokerite, cetostearyl alcohol, stearyl alcohol, cetyl alcohol, myristyl alcohol, glyceryl monostearate, glyceryl palmitostearate, glycerol monooleate, glyceryl behenate, cetyl esters, acetylated monoglycerides, tristearin, tripalmitin, hydrogenated vegetable oils, and a combination thereof.

28. The pharmaceutical composition of claim 1, 2, 3 or 4, wherein the sustained release agent is polyacrilin potassium.

29. The pharmaceutical composition of claim 1, 2, 3 or 4, wherein the composition contains sodium bicarbonate.

30. The pharmaceutical composition of claim 1, 2, 3 or 4, wherein the sustained release agent is selected from the group consisting of poly(methyl methacrylate); poly(ethyl methacrylate); poly(methyl acrylate); poly(isopropylacrylate); poly(isobutylacrylate); poly(octadecyl acrylate); ethyl cellulose; cellulose propionate; cellulose acetate propionate; and a combination thereof.

31. The pharmaceutical composition of claim 1, 2, 3 or 4, wherein the sustained release agent is selected from the group consisting of a wax; a fatty alcohol; a fatty acid ester; hydrogenated vegetable oil; and a combination thereof.

32. A pharmaceutical composition comprising:
a first release portion and a second release portion, wherein the first release portion and the second release portion each comprise an active agent selected from the group consisting of atomoxetine, a pharmaceutically acceptable salt of atomoxetine, and a combination thereof;
wherein the first release portion is an immediate release portion and the second release portion is an extended release portion and the second release portion releases the active agent at a slower rate than the release rate of the first release portion in an in vitro dissolution test; and
wherein the composition releases: at least about 20% of the total weight of the active agent by about 2 hours, at least about 25% but less than about 70% of the total weight of the active agent by about 4 hours, at least about 40% but less than about 90% of the total weight of the active agent by about 6 hours, and at least 90% of the total weight of the active agent by about 12 hours, as measured by an in vitro dissolution test.

33. The pharmaceutical composition of claim 32, wherein the active agent is present in the first release portion in an amount of about 20% to about 40% of the total weight of the active agent in the composition, and the active agent is present in the second release portion in an amount of about 40% to about 80% of the total weight of the active agent in the composition.

34. The pharmaceutical composition of claim 32, wherein the active agent in the first release portion and in the second release portion is atomoxetine HCl, and wherein the amount of active agent in the first release portion is equivalent to about 6 mg of atomoxetine, and the amount of active agent in the second release portion is equivalent to about 12 mg of atomoxetine.

35. A method for treating or reducing the incidence of orthostatic hypotension (OH) in a subject in need thereof comprising administering the pharmaceutical composition of claim 32 to the subject.

36. A method for treating or reducing the incidence of postural orthostatic tachycardia syndrome (POTS) in a subject in need thereof comprising administering the pharmaceutical composition of claim 32 to the subject.

37. A method comprising administering the pharmaceutical composition of claim 32 to a subject to treat or reduce the incidence of a condition selected from the group consisting of: orthostatic hypotension, postural orthostatic tachycardia syndrome (POTS), vasovagal syncope, dysautonomia, retrograde ejaculation or other disorder of semen ejaculation, symptoms of chronic orthostatic hypotension corresponding to autonomic failure associated with Bradbury-Eggleston, Shy-Drager syndromes, diabetes mellitus disease, and Parkinson's disease; in the subject.

38. A method for treating or reducing the incidence of vasovagal syncope in a subject in need thereof comprising administering the pharmaceutical composition of claim 32 to the subject.

39. A kit comprising a first formulation and a second formulation, wherein the first formulation comprises a pharmaceutical composition of claim 1, 2, 3 or 4, and the second formulation comprises a second active agent.

40. The kit of claim 39, wherein the second active agent is selected from the group consisting of hydrocortisone, fludrocortisone, octreotide, pharmaceutically acceptable salts thereof, and a combination thereof.

41. The pharmaceutical composition of any of claim 1-15 or 24-34, wherein the in vitro dissolution test is performed with USP Apparatus I (baskets) at 100 rpm in 900 mL at 37° C., 0-2 hours, 0.1N HCl (pH 1.2); 2-4 hours, acetate buffer (pH 4.5); 4-12 hours, phosphate buffer (pH 6.8).

42. The pharmaceutical composition of claim 1, 2, 3, 4 or 32 for use in treating orthostatic hypotension in a subject in need thereof.

43. The pharmaceutical composition of claim 1, 2, 3, 4 or 32 for use in reducing the incidence of orthostatic hypotension in a subject in need thereof.

44. The pharmaceutical composition of claim 1, 2, 3, 4 or 32 for use in treating a condition selected from the group consisting of: orthostatic hypotension, postural orthostatic tachycardia syndrome (POTS), vasovagal syncope, dysautonomia, retrograde ejaculation or other disorder of semen ejaculation, symptoms of chronic orthostatic hypotension corresponding to autonomic failure associated with Bradbury-Eggleston, Shy-Drager syndromes, diabetes mellitus disease, and Parkinson's disease; in a subject in need thereof.

45. The pharmaceutical composition of claim 1, 2, 3, 4 or 32 for use in reducing the incidence of a condition selected from the group consisting of: orthostatic hypotension, postural orthostatic tachycardia syndrome (POTS), vasovagal syncope, dysautonomia, retrograde ejaculation or other disorder of semen ejaculation, symptoms of chronic orthostatic hypotension corresponding to autonomic failure associated with Bradbury-Eggleston, Shy-Drager syndromes, diabetes mellitus disease, and Parkinson's disease; in a subject in need thereof.

46. The pharmaceutical composition of claims 42, 43, 44 and 45, wherein the active agent in the composition is atomoxetine HCl, and the total amount of active agent in the composition is equivalent to an amount of atomoxetine selected from the group consisting of about 10 mg, about 18 mg, about 40 mg, about 80 mg and about 100 mg.
